(19) United States
(12) Patent Application Publication
(10) Pub. No.: US 2026/0358283 A1
(43) Pub. Date: Nov. 12, 2026

(54) SYSTEMS AND METHODS FOR QUALITY VERIFICATION FOR A MIXTURE

(71) Applicant: 3M INNOVATIVE PROPERTIES COMPANY, St. Paul, MN (US)

(72) Inventors: Joerg Hahn, Bad Neuenahr-Ahrweiler (DE); David M. Rudek, Duesseldorf (DK); Knut Schumacher, Neuss (DE); Waleri Wischnepolski, Neuss (DE); Michael H. Stalder, Uedem (DE); Ryan P. Marrinan, Lino Lakes, MN (US); Benjamin J. Muenstermann, Duisburg (DE)

(21) Appl. No.: 19/135,128

(22) PCT Filed: Dec. 8, 2023
(86) PCT No.: PCT/IB2023/062401

§ 371 Date: Jun. 3, 2025

Related U.S. Application Data
(60) Provisional application No. 63/386,715, filed on Dec. 9, 2022.
(60) Provisional application No. 63/486,631, filed on Feb. 23, 2023.
(60) Provisional application No. 63/507,662, filed on Jun. 12, 2023.

(51) Int. Cl. G01N 27/07; G01N 27/04; G01N 27/10; G01N 27/20; G01N 27/22; G01N 27/24

(52) U.S. Cl. CPC G01N 27/07; G01N 27/045; G01N 27/10; G01N 27/20; G01N 27/226; G01N 27/228; G01N 27/24

(57) ABSTRACT

A sensing system for a mixture includes a sensing zone containing a mixture and a sensor within the sensing zone. The sensor includes a laminate structure including an insulated layer, a conductive layer and a conductive trace. The sensor includes a first sensing area and a second sensing area, the first sensing area is configured to operate as a transmitting or receiving electrode. The first and sensing areas are arranged in line with the sensing zone such that the mixture directly contacts both the first and second sensing area. A sensor signal is generated at the receiving electrode when an electric field is provided at the transmitting electrode. The system includes a communication component that communicates an electrical parameter signal based on the sensor signal.

### BACKGROUND

Many products require mixtures in manufacture—e.g. paint for commercial or industrial use, adhesives, resins, etc. Many mixtures include different materials that, over time, may settle or separate. It may not be easily recognized by a user of the mixture that the composition is no longer consistent.

### SUMMARY OF THE DISCLOSURE

A sensing system for a mixture includes a sensing zone containing a mixture and a sensor within the sensing zone. The sensor includes a laminate structure including an insulated layer, a conductive layer and a conductive trace. The sensor includes a first sensing area and a second sensing area, the first sensing area is configured to operate as a transmitting or receiving electrode. The first and sensing areas are arranged in line with the sensing zone such that the mixture directly contacts both the first and second sensing area. A sensor signal is generated at the receiving electrode when an electric field is provided at the transmitting electrode. The system includes a communication component that communicates an electrical parameter signal based on the sensor signal.

Systems and methods including such sensors allow for direct contact between the sensor and a fluid flowing through a dispenser as sensors herein are cost effective to manufacture and can be discarded after use. Systems and methods herein also allow for multiple sensor signals to be gathered across a fluid flow, providing real-time information about materials going into, and out of, a mixing area. Systems and methods herein also allow for bubble detection and removal. Systems and methods herein allow for dispensing systems and their operators to change operational parameters during an operation to address issues as they are occurring, or potentially before the occur, such that less material is wasted and more accurate dispensing is possible.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list. Thus, the scope of the present disclosure should not be limited to the specific illustrative structures described herein, but rather extends at least to the structures described by the language of the claims, and the equivalents of those structures. Any of the elements that are positively recited in this specification as alternatives may be explicitly included in the claims or excluded from the claims, in any combination as desired. Although various theories and possible mechanisms may have been discussed herein, in no event should such discussions serve to limit the claimable subject matter.

### BRIEF DESCRIPTION OF FIGURES

FIGS. 1A-1C illustrate systems for dispensing an atomized fluid that may benefit from systems and methods herein.

FIG. 2 illustrates an exploded view of a spray gun, in which embodiments described herein may be implemented.

FIGS. 3A-3D illustrate material measurement flow sensors in accordance with embodiments herein.

FIGS. 4A & 4B illustrate material measurement flow sensors as used in accordance with embodiments herein.

FIGS. 5A-5D illustrate example implementations of dispensing systems with sensor systems installed in accordance with embodiments herein.

FIGS. 6A-6C illustrate a sensor configuration for bubble detection in accordance with embodiments herein.

FIGS. 7A-7C illustrate a sensor configuration in accordance with embodiments herein.

FIGS. 8A-8C illustrate electrical schematics for sensing systems in accordance with embodiments herein.

FIGS. 9A-9B illustrate sensor configurations in accordance with embodiments herein.

FIG. 10 illustrates a method of quality controlling a material dispensing system in accordance with embodiments herein.

FIG. 11 illustrates a quality control system, in accordance with embodiments herein.

FIG. 12 illustrates a method of receiving real-time sensor signals in accordance with embodiments herein.

FIGS. 13A-13C illustrates conductivity measurement system in example network architectures.

FIGS. 14A-14D illustrates a control system for an electrical parameter sensor in accordance with embodiments herein.

FIGS. 15-18 illustrates example computing devices that can be used in embodiments herein.

### DETAILED DESCRIPTION OF ILLUSTRATIVE EMBODIMENTS

The present disclosure relates to systems and methods that include sensors that determine properties of fluids in-situ. The disclosure also relates to data sets received by such sensors and methods of using said data for analyzing said fluid properties. Using systems and methods described herein, it may be possible to adjust use conditions of a mixture (e.g. change pressure, temperature, mix ratio, etc.) or to improve composition consistence (e.g. re-mix, off-gas, etc.) before, or during, an operation.

Many industrial processes use mixtures such as liquid adhesives, liquid food ingredients, liquid coolants, or liquid reaction products, to name a few examples. Certain properties of such liquids vary over time: a dispersion or emulsion may separate, an oil may become less viscous as temperature rises, a coolant may age and have a lower heat capacity than initially. Performance of the product as used may suffer. For example, paint may have a soft cure (or no cure at all), may be brittle, crack, experience delamination or poor adhesion. If a paint mixture is not consistent before application, corrective action may take considerable time-energy-intensive sanding and surface preparation may be required before the painting operation is attempted a second time. Troubleshooting these issues require detailed chemical knowledge, time and elimination of other causes. For many operations, troubleshooting costs time that cannot be spared.

Co-pending international application IB2021/056362, filed on Jul. 14, 2021, discloses a property sensor for determining a property value of a liquid that includes two PCB boards that define a channel through which the liquid flows. While this allows for direct contact between the sensor and the fluid, there exists a need for cost-effective sensors that can provide more contextual information about material mixing. Embodiments herein provide systems and methods for effectively and accurately measuring material information for mixture quality control.

Described herein are sensors and sensor systems that are used to measure electrical properties of fluids. Broadly sensors herein function by a transmitting electrode, using a provided current or voltage, creates an electrical field. As a fluid flows between the transmitting electrode and a receiving electrode, it conducts a current to the receiving electrode. The term “sensor” as used herein may refer both to the physical sensor that provides a sensor signal indicative of conducted current, as well as to a “sensor system” that includes a processor that calculates an electrical property of the fluid based on the sensor signal.

The term “electrical property” is intended to broadly refer to any electrical property of a fluid that can be derived based on impedance measurements of a sensor. Used herein, for ease of understanding the embodiments, are the example of impedance measurements. However, it is expressly contemplated that other electrical properties may be calculated and relevant to embodiments herein. For example, conductivity measurements or dielectric constants may also be determined from impedance measurements. Either conductivity or dielectric constant may be relevant, as illustrated herein, for determining relevant functionality of a dispensing system or quality of fluids flowing therein.

As used herein, the term “real-time” refers to data is processed within milliseconds so that it is available virtually immediately as feedback. While some delay due to processing are inevitable, “real-time” is intended to cover systems and methods where data can be collected or entered and a user can then interact with it without noticeable delay. E.g. a user may make a data entry into a system, and the data entry is then substantially immediately available for viewing or editing.

As described herein, sensors are described as measuring electrical properties of “fluids.” The term “fluid” is intended to be interpreted broadly and is intended to cover liquids with low viscosities, liquids with high viscosities, semi-solid materials, suspensions, melted materials, or other flowable materials.

Electrical parameters, as used herein, may be detected by an electrode pair. Fluid may flow between or past the electrode pair. A transmitting electrode may generate an electric field when a voltage or a current is applied, while a receiving electrode receives a current or voltage. The sensed electrical parameter may be a conductivity, relative permittivity or an impedance. The terms relative permittivity and dielectric constant are used herein interchangeably.

Sensors are described herein as having one or more “apertures” within a “printed circuit board.” These terms are intended to be interpreted broadly. For example, an aperture may fully extend through a thickness of a sensor along part of, or the entirety of its length. Apertures may have beveling along part or all of a perimeter. An aperture may be elongated, such as a slot, or may be shaped, such as a circular or ovular hole. An aperture may have one or more corners or edges, or may have curvature along part or all of its perimeter. As used herein, a “printed circuit board” refers to a laminated sandwich structure of conductive and insulating layers. Printed circuit boards (PCBs) herein may include any number of terminals and conductors that allow for voltage to be applied to a transmitting electrode and for current to be transmitted from a receiving electrode. Alternatively, PCBs may also be constructed to allow for a current to be applied and voltage transmitted. PCBs may be manufactured using traditional PCB manufacturing technology or additive manufacturing technology. As used herein, PCB is intended to cover any number of layers, with or without an edge connector. Any suitable conductive metal may be used to form conductive layers. Any suitable insulating material may be used to form insulating layers.

Property sensors as described herein may be used to sense properties of a fluid resulting from a mixing process. They may also be used to sense properties of input fluids for a mixing process or for an industrial manufacturing process. Advantageously, separate property sensors for respective input fluids are placed just in front of the mixer. Data from these property sensors measuring the input fluids can be processed along with data from a property sensor measuring the mixed fluid, e.g. in an integrated materials property monitoring system. Where, for example, a fluid composition is mixed from three input fluids, a property of each of the three fluids before mixing can be determined using three property sensors at the respective outlets of the three containers containing the three input fluids. This may help in quality control and reduce waste that might otherwise occur due to one of the input fluids being outside a specification for the property.

Sensors described herein may determine various properties of a fluid, like, for example, mixing ratio of a two-component adhesive or curing status of a curable composition or ageing status.

The term “curing” as used herein is intended to broadly cover a changing of a material from a first state to a second state. For example, some liquids cure into solids. Some mixtures may experience crosslinking. Some mixtures may experience pre-polymerization. Some mixtures may experience conversion. The number of properties which were varied previously to establish the set of calibration data representing calibration impedance responses measured previously at the different property values determines the number of properties that can later be determined by the property sensor. The pre-stored set of calibration data representing calibration impedance responses measured previously at the one or more sensing frequencies and at different property values of a property of the fluid forms, or represents, a multi-dimensional data field which is specific for the fluid. This data field allows the property value deriver to determine, from a response impedance actually measured, a value of the property of the fluid.

A fluid has many properties: for example, viscosity, density, color, content of volatile components, water content, chemical composition, boiling point, but also ageing status, curing status in case of fluid curable compositions, or mixing ratio in case of the fluid being a mixture, to name only some.

Further, certain properties of certain fluids, however, vary with time and/or with other parameters such that the response impedance in a property sensor described herein varies with time and/or with the other parameters, too. Values of these properties may be derived via sensors and systems described herein. Additionally, variation with time includes variation of the property between different production lots of the fluid. The property sensor described herein can thus be used to detect differences in a certain property (e.g. chemical composition) of a suitable fluid between a later production lot and an earlier production lot of the fluid.

The term “property” of the fluid, according to the present disclosure, is not particularly limited. For example, as described in embodiments herein, one property of interest is a mixing ratio of two or more components of the fluid. In certain of these embodiments, the fluid is a two-component adhesive, and a property of the fluid is a mixing ratio of the components. In other embodiments, a property of interest is a curing degree or a curing status. In certain of these embodiments the fluid is a curable composition, and a property of the fluid is the degree of curing of the composition.

In other embodiments, a property of interest is an ageing degree or an ageing status. In certain of these embodiments the fluid is an ageing fluid, i.e. a fluid in which certain characteristics change over time once the ageing fluid has been created. The property sensor may determine a change in the response impedance of the ageing fluid after some ageing, compared to response impedances of an identical fluid recorded before ageing and at certain times after ageing. The property sensor may thereby determine an ageing degree or an ageing status of the fluid.

A property of the fluid may take different values, such as, for example, a property “dynamic viscosity” of the fluid “water” can take values like 1.30 mPa·s or 0.31 mPa·s. Such values are referred to herein as property values. Certain properties may not be related to only numerical property values. A property “curing degree”, for example, may have property values like, for example, “uncured”, “partially cured” or “fully cured”. A property “curing status”, for example, may have property values like, for example, “uncured” or “fully cured”. A fluid according to the present disclosure may be a viscous fluid. Independent of its viscosity, the fluid may be a flowing fluid. The fluid may be a continuously flowing fluid.

“Fluid” or “fluid mixture” are used broadly herein to refer to a composition comprising two or more components. The components may both be liquids, or it may be particulates in a liquid, etc. Generally, a “fluid” or “fluid mixture” refers to a flowable substance. Systems and methods herein may be useful for a range of fluid applications including, but not limited to: paint, resin-for adhesive or other purposes, cure-in-place gaskets, adhesives or other coating materials, dental impression material, void filler, sealant, an engineered fluid, a thermally conductive interface material, a precursor material to any of these, or emulsions or any material that can lose stability over time.

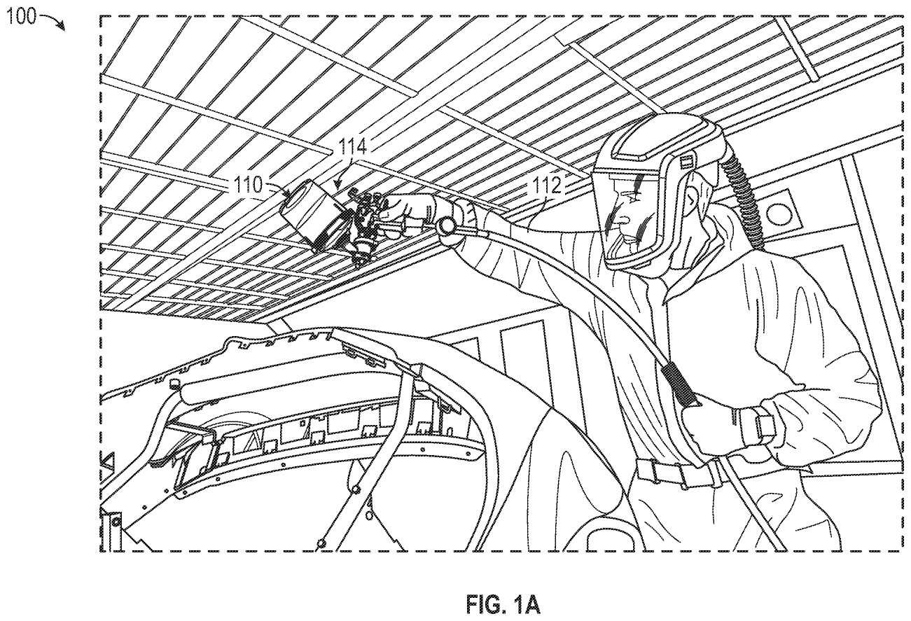

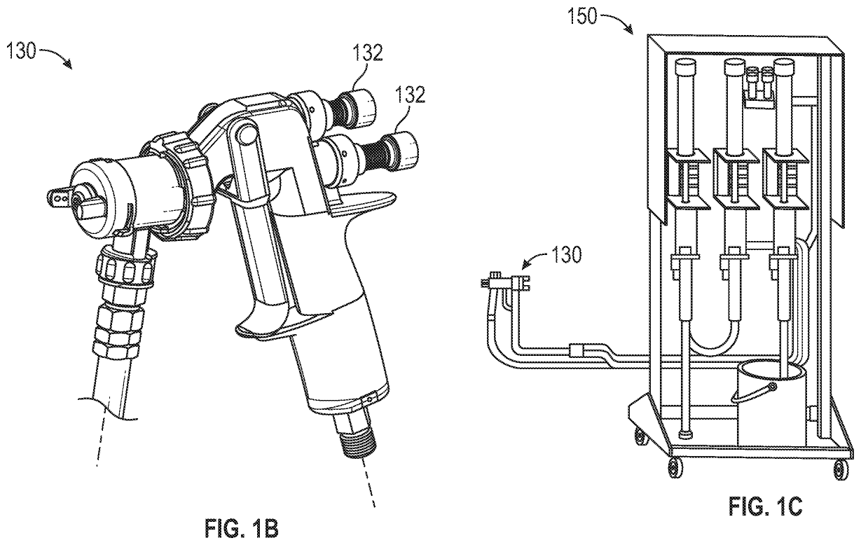

FIGS. 1A-1C illustrate systems for dispensing an atomized fluid that may benefit from systems and methods herein. FIG. 1A illustrates a painting operation 100 where a spray gun 114 atomizes paint from a paint cup 110 using air from air supply 112. However, while a painting operation is illustrated, container 110 could provide other material for dispensing. FIG. 1B illustrates another configuration of a spray gun 130, that receives two materials and provides an atomized mixture. Spray gun 130 may be coupled to a system 150, illustrated in FIG. 1C. System 150 may include pumping systems for one or both components 132, and/or a pressurized air source.

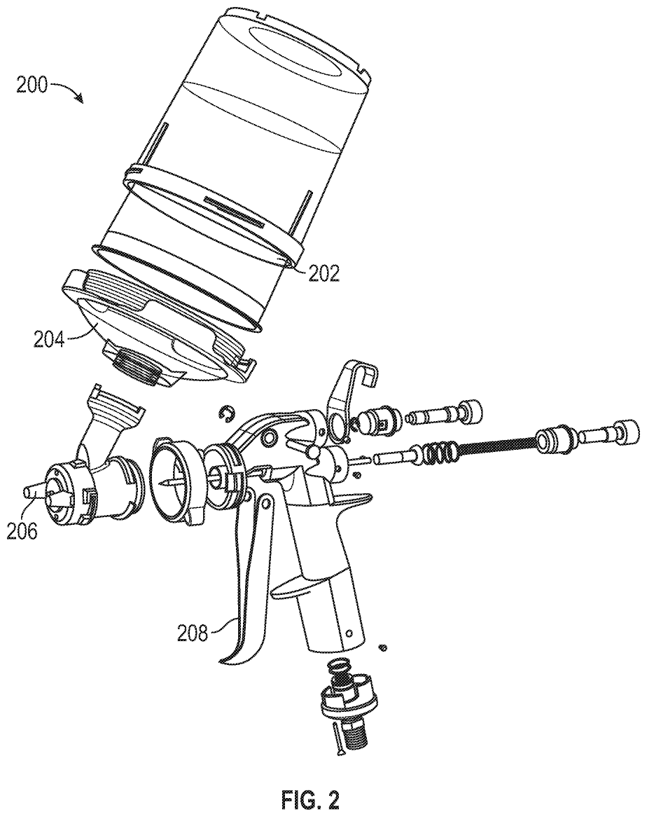

FIG. 2 illustrates an exploded view of a spray gun, in which embodiments described herein may be implemented. Spray gun 200 includes, among other features, includes a container 202 that holds fluid to be dispensed. However, while a container 202 is illustrated that couples to a nozzle using fastener 204, it is expressly contemplated that a larger container, may feed fluid to spray gun 200, for example using a pump. Spray gun may actuate when trigger 208 is pulled, for example.

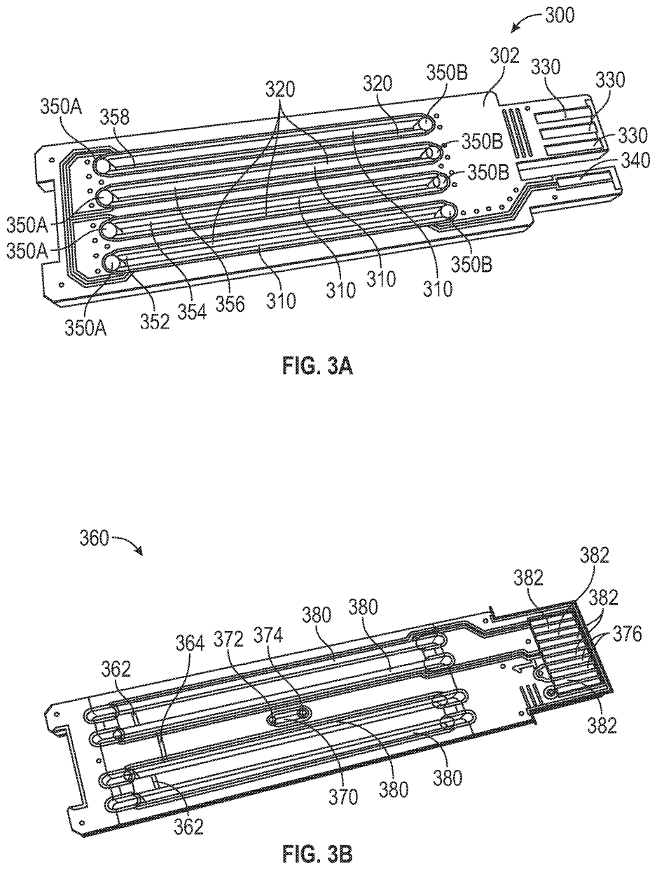

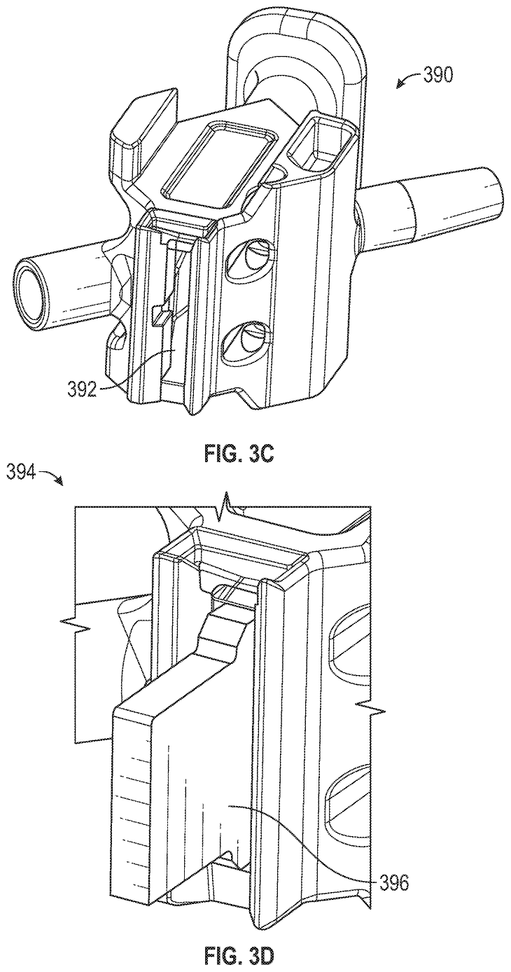

FIGS. 3A-3D illustrate material measurement flow sensors in accordance with embodiments herein. FIG. 3A illustrates a PCB material measurement flow sensor 300. As illustrated in FIG. 3A, a sensing system 300 includes a PCB board 302 with one or more grounds 330 and a TX contact 440. The TX contact provides a transmitting signal to each transmitting electrode 310. Four RX contacts (not shown), located on the reverse side of the PCB, receive the indication of a sensed impedance from each of the electrode pairs. The electrical potential of each receiving electrode 320 is electronically regulated to ground potential separately. The regulator action for each receiving electrode, in some embodiments, is interpreted as an impedance signal for each electrode pair. In the illustrated embodiment, four separate measurements channels can provide information, each through its own TX contact 340 and RX contact (not shown).

In the illustrated embodiment, a sensing system 300 has four electrode pairs, with four transmitting electrodes 310, each paired with one of four receiving electrodes 320. However, it is expressly contemplated that more, or fewer, electrode pairs may be present, depending on available area on a PCB board and sensing needs.

Each of the electrode pairs are decoupled from the adjacent pair such that four separate conductivity measurements are received, one from each electrode pair 310, 320. Sensing system 300 is placed, in some embodiments, perpendicularly to the flow of material, such that a first sensing area 352 receives a first portion of material flow, a second sensing area 354 receives a second portion of material flow, a third sensing area 356 receives a third portion of material flow, and a fourth sensing area 358 receives a fourth portion of material flow. Therefore, system 300 can simultaneously generate four different signals relative to a single material flow, providing a better picture of whether a mixing ratio (or other measured parameter) is consistent across an entire sensing area.

In comparison to previous sensing systems, conductivity measurements required both a positive and a negative pole, which would require two PCBs per electrode pair. In contrast, system 300 allows for four measurements to be taken simultaneously with a single PCB. It also provides a larger surface area for material flow, through a shorter sensor distance.

FIG. 3A illustrates an embodiment where each electrode pair is part of a slot 352, 354, 356, 358. However, it is also contemplated that, instead of being closed on both sides, a sensing area may include a pair of electrodes on a protrusion, or within an aperture, in a “comb”-like structure. However, it may be preferred for both ends to be closed from a structural standpoint, especially with viscous fluids.

As described further herein, the electrodes 310, 320 may be formed by metallization on the interior surface of slides 352, 354, 356, 358, using copper for example. The metallization process may cause electrodes 320 to be connected to electrodes 410. Therefore, a decoupling or disconnecting step is needed. This can be done by breaking the connection, for example by drilling a hole in the positions 350A and 350B as illustrated, by punching out a perforated component, milling, nibbling, etching, laser cutting or another suitable method.

Systems and methods herein may be used for a variety of materials being dispensed. PCB boards often have a maximum operating temperature less than 170 °C, which limits the temperature of materials that can be dispensed through a sensor system 300. Materials may have a range of viscosities, for example up to around 10⁵ Pa s. Higher viscosity might result in a dispensing pressure being insufficient to force the material through slots 352-358 without breaking the sensor. However, higher viscosity materials may be accommodated by increasing the width of slots 352-358. However, sensing system 300 may be less sensitive. Similarly, for materials with particulates, such as suspensions for example, particle sizes have to be smaller than the width of slots 352-358. Additionally, systems herein may be limited to solvents that do not cause corrosion or otherwise damage the PCB 302 or electrodes 310, 320.

FIG. 3B illustrates another embodiment of a sensing system 360, which includes a built-in temperature sensor 370. Temperature sensor 370 sits within a slot with a connection point 372 for a ground signal and a connection point 374 for a temperature signal. Ground signal connection point 372 connects to a ground signal communicator 382. Temperature signal communication point 374 connects to a temperature signal communicator 376. Similar to the embodiment of FIG. 3A, four impedance or conductivity sensor slots 380 are also present, each connected to a ground signal 382. However, it is noted that two different spacings between slots are present in the embodiment of FIG. 3B. A first spacing, 362 is present between a first and second slot 380, and between a third and fourth slot 380, while a second spacing 364 is present between second and third slots 380. Increased spacing 364 may provide improved shielding against interference between electromagnetic fields generated by each electrode pair.

Many mixing processes are at least partially temperature dependent, with material properties like viscosity changing with temperature. Temperature sensors inserted from an external point are often fragile and need to be in the middle of the flow of the material being tested. In the embodiment of FIG. 3B, a temperature sensor is sealed within a housing, which keeps it isolated from the material. The seal layer may be a layer of varnish, for example, which may allow for the thermal contact to be improved relative to other housing materials. As illustrated, the temperature sensor connects via contacts 382 on the edge connector.

FIGS. 3A-3B illustrates an embodiment where slots 352-358, 370 and 380 are ovular in shape, with a generally straight body and rounded ends. However, other configurations are possible. Electrodes 310, 320 may be curved, for example, or otherwise shaped to accommodate an available volume of a dispensing system.

While FIGS. 3A-3C illustrate embodiments where each slot 352-358 comprises a single electrode terminal, it is expressly contemplated that in some embodiments one or more slots 352-358 may house multiple electrode terminals—e.g. with one or more terminals along the length of one or more of slots 352-358. Having a third or fourth terminal may allow for more accurate measurement of an electrical parameter. Instead of a configuration where the circuit configuration includes an ohmmeter that measures a resistance, there is instead an ammeter and a voltmeter. The voltmeter measures a voltage across the circuit, while the ammeter is measuring the current flowing through the circuit. An impedance (e.g. resistance) can be derived from the measured voltage and current. Such a setup may result in more accurate measurements of electrical parameters within a fluid flowing through slots 352-358.

FIGS. 3C-3D illustrate a housing for a sensor in accordance with some embodiments herein. A sensor, such as sensor 300 or 360 may be directly received by a material dispensing system in some embodiments. However, it is also contemplated that, in some embodiments, a housing 390 may receive a sensor 396 directly. Housing 390 includes a receiving slot 392 that receives a sensor, as illustrated in configuration 394.

In some embodiments, housing 390 is built into a dispensing system such that a sensor 396 is received by the dispensing system. In some embodiments, a dispensing system receives housing 390, with sensor 396 already installed therein. Sensor 396 may be sealed into housing 390, in some embodiments, such that a dispensing system receives housing 390.

Using systems and methods described herein, it is possible to monitor a number of parameters relevant to the quality of a mixture prior to, or during use of said mixture. Monitoring mixture quality may refer to any of consistency, texture, composition or other relevant quality indication. Sensor systems and methods of use herein may provide indications of mix ratio, curing (e.g. open time, curing speed, temperature changes) and may provide in-situ process indications such as aging, air bubble detection or concentration, lot-to-lot variation, raw material quality, and phase separation. Using sensor systems herein, it is possible to automatically detect a quality concern (mix ratio imbalance, phase separation, etc.) and provide indications for correcting the quality concern, so that a correction can occur in-situ.

Early detection of quality concerns can help reduce correction time and will, therefore, reduce operation time, corrective supply cost, and corrective operation time and cost. Sensors described herein can be implemented in many parts of a dispensing operation—at intake, during or after mixing, within a dispenser, within a container, etc.

Sensors described herein are communicable with a computerized control system which may provide an alternating current (AC) voltage to generate a required electric field needed for measuring conductivity, impedance or dielectric constant using a suitable sensing system, such as that described herein. The control system may also, in some embodiments, provide a current. While various examples of this disclosure are described with respect to the use of AC, it will be appreciated that techniques of this disclosure may be performed using direct current (DC) in other examples.

When running an actual measurement of quality control parameters of a mixture, the measured impedance responses (MIR), each measured at certain measurement sensing frequencies (MSF), can be recorded in the control system.

In order to derive a value for the mixing ratio, for example, from the measured impedance responses at the measurement sensing frequencies, software running on the control system identifies, within the set of calibration impedance response triples, those triples having the closest calibration response impedances, closest to the measured impedance responses, and the closest calibration sensing frequencies, closest to the measurement sensing frequencies. This identification and a potential interpolation can be performed easily by using the parametrized multi-dimensional polynomials modelling the plurality of data sets, i.e., the plurality of triples of (CMR, CSF, CIR). From those calibration data, the software derives a value for the (so far unknown) mixing ratio in the actual measurement.

The same sensing frequencies used for calibration will often be used also for the measurement. There may, however, occur a mixing ratio in the measurement for which no calibration impedance response had been determined in calibration. So there may be not an exact match in both sensing frequency and response impedance between a triple in the calibration data set. In such a case, an interpolation between two suitably chosen calibration triples, containing two calibration impedance responses close to the measured response impedance, yields an interpolated calibration mixing ratio which can then be considered the mixing ratio in the measurement. The interpolation is performed by software on the control system 220, using the parametrized multi-dimensional polynomials.

The result of the interpolation and derivation is a value of the mixing ratio of components A and B as the mixture passes through the PCB sensor during the measurement.

In the present embodiment, the calibration impedance responses were measured in their dependence on two parameters, namely on the sensing frequency and on the mixing ratio. In other embodiments, dependence of impedance responses on further parameters may be taken into account, such as, for example, dependence on the temperature of the adhesive in the sensing zone. A data set of the calibration impedance responses would then be a quadruple of values, such as (CMR, CSF, CIR, Temperature), and the pre-stored set of calibration impedance responses would be a set of quadruples forming a four-dimensional data field, which is specific for the mixture. Taking further parameters into account could make a data set be a quintuple of values, or high-order tuples of values, so that the data sets of calibration impedance responses is a multi-dimensional data field of more dimensions and can be represented by different parametrized multi-dimensional polynomials.

A control system may record the values for mixing ratio, with a time stamp, for quality assurance. The mixing ratio derived during the actual measurement can be checked continuously against a desired mixing ratio. If its deviation from the desired mixing ratio is larger than acceptable, the control system may change the flow rate of either components suitably to adjust the measured mixing ratio towards the desired mixing ratio.

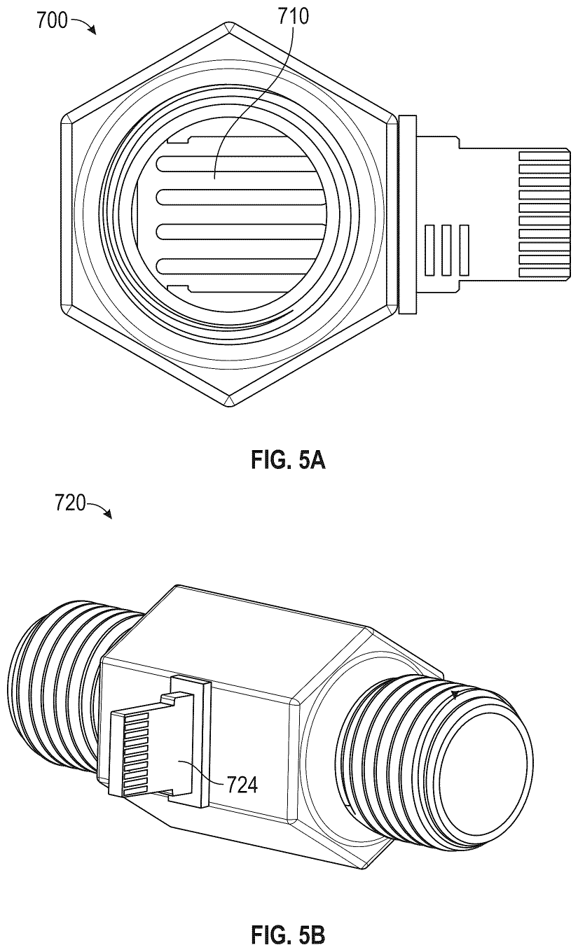

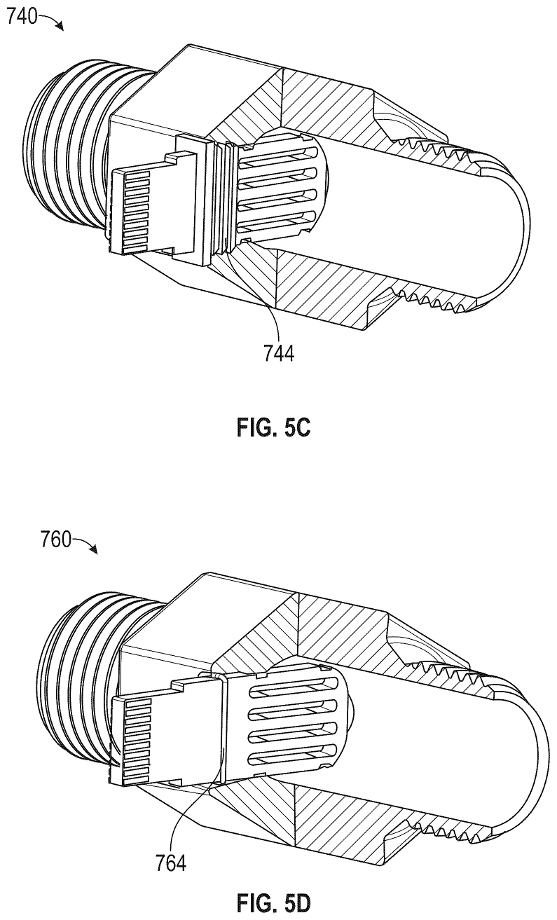

A method of forming sensor systems like those illustrated herein may be similar to that described in PCT/US22/52343, for example FIG. 5 and the associated description, which is incorporated herein by reference.

FIGS. 5A-5B illustrate material measurement flow sensors as used in accordance with embodiments herein. As illustrated in both images, sensors according to embodiments herein can be placed in direct contact with a material or fluid, providing a conductivity measurement based on that direct contact. This provides a more accurate measure of mixing ratio than other methods that do not allow for direct contact between a sensor and a material. However, as illustrated in FIGS. 5A and 5B, a sensor is coated with material after use. In scenarios where the material of interest is corrosive, highly viscous or is curable, it is beneficial to be able to discard the sensor after use.

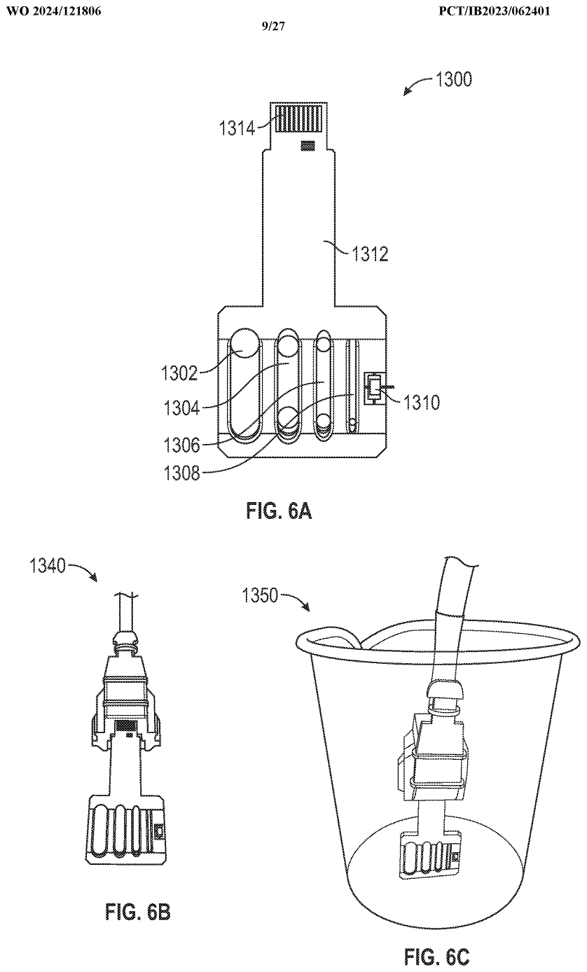

FIGS. 6A-6C illustrate a sensor configuration that may be particularly useful to detecting bubbles or aggregation of droplets of a second phase forming prior to a phase separation.

FIG. 6A illustrates a dip sensor that may be particularly useful for detecting air bubbles or droplets in a mixture. Sensor includes four electrode pairs in four slots, 1302, 1304, 1306 and 1308, that are different sizes. Slot 1302 is wider than slot 1304, which is wider than slot 1306, which is wider than 1308. Slots 1302-1308 are illustrated in an arrangement from thickest to thinnest, however it is expressly contemplated that other arrangements are possible. Similarly, while four coplanar electrode pairs are illustrated, all substantially the same distance from a connection end of a sensor—e.g. the portion that connects to a signal reader. An edge connector 1314 is shown as one example, however other suitable connections may be possible.

Slots 1302-1308 are designed to both detect bubbles or droplets and provide an indication of size. Generally, a consistent mixture with no bubbles or droplets provides an insulation effect, and maintain a consistent conductivity across all electrode pairs. When a droplet reaches a width of one of the slots, the droplet will connect both sides of the electrodes, resulting in a detectable change in conductivity.

The design illustrated in FIG. 6A is illustrated as having slot widths that linearly increase e.g. 1 mm, 2 mm, 3 mm and 4 mm. A linear increase in diameter corresponds to a cubic increase in volumetric flow through the apertures. Such a configuration provides a good understanding of how quickly phase separation will occur and/or how stable a mixture is. For example, if phase separation is more than a hour away, it may still be possible to dispense a mixture without corrective action.

However, it is explicitly contemplated that some embodiments may require smaller or larger slot sizes. For example, a thinnest slot may be as thin as 100 μm, or thinner than 150 μm, or thinner than 200 μm, or thinner than 300 μm, or thinner than 400 μm. One or more slots may be thinner than 500 μm. One or more slots may be thinner than 1 mm. For other applications, for example a stir stick being used in a larger measurement operation, such as checking a mixture quality of a 50 gallon drum.

In addition to the change in width, the slots may also change in length to suite a particular application. For example, in checking the shelf-life of a larger container, the overall sensor may need to be much longer—for example up to, or over, 1 meter in length. In such instances, apertures must be larger—both to increase signal strength and to allow for significant flowthrough. A length may be increased to increase signal strength, balanced with a width selected to allow flowthrough without sacrificing signal strength. For example, for a meter-long sensor, the dimensions may be 10 centimeters long and 1 cm wide.

E.g. when a droplet (or bubble) reaches a diameter as wide or wider than slot 1308 (the narrowest slot), it connects the two electrodes within slot 1308, resulting in a conductivity spike only for electrode pair 1308, as, until the droplet (or bubble) diameter grows to a width as wide or wider than slot 1306, it will not connect the two sides of slot 1306. The conductivity will return to a baseline for the mixture once the bubble (or droplet) passes through slot 1308. Depending on the number of droplets (bubbles) in the mixture, the conductivity spike frequency changes.

Sensor 1300 also includes a temperature sensor 1310, illustrated as in-line with electrode pairs 1302-1308, it is expressly contemplated that temperature sensor 1310 may be positioned in another suitable position. Additionally, it is contemplated that, for some embodiments, a temperature sensor 1310 is not needed, e.g. for a mixture that does not change viscosity significantly over a temperature range of use.

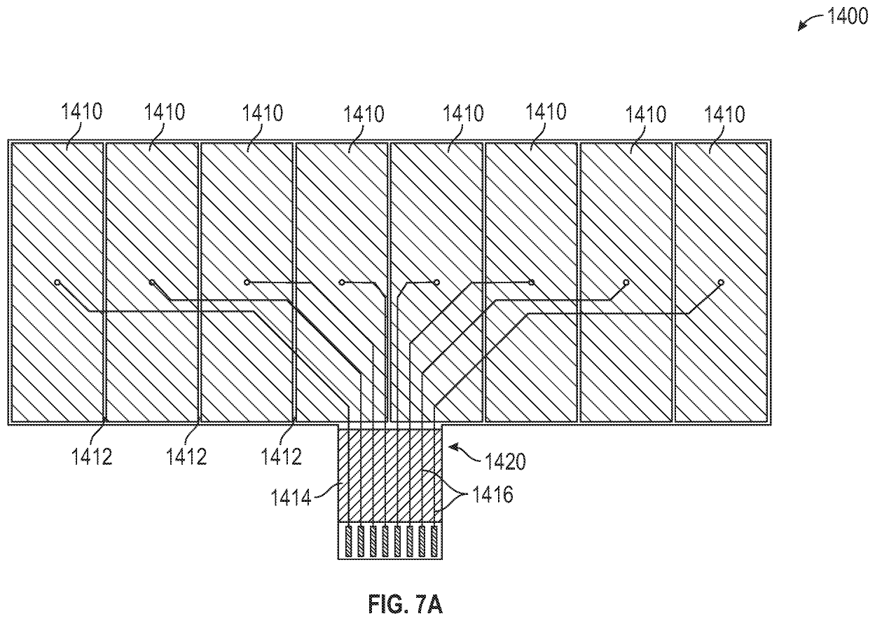

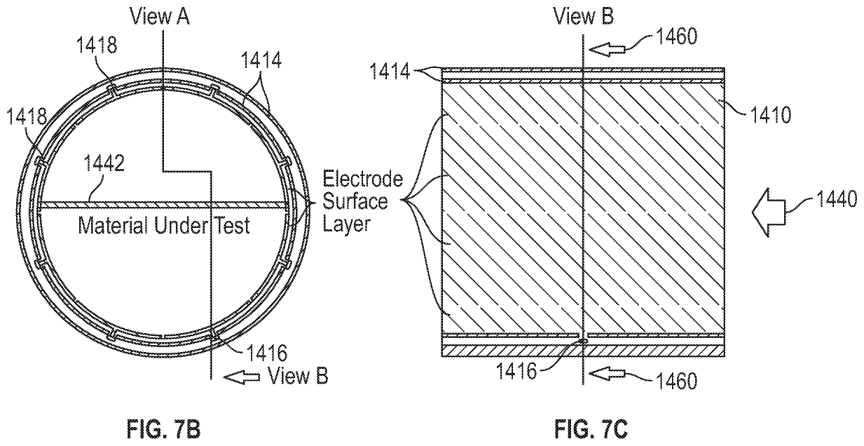

Sensor 1300 is also illustrated as having a length 1312 that separates the edge connector 1314 from electrode pairs 1302-1308. However, length 1312 may not be necessary if sensor 1300 is used as an in-line flow sensor, for example mounted within a conduit as illustrated in FIGS. 7A-6D.

FIGS. 6B and 6C illustrates a sensor connected to lead wires 1340, illustrating how a length 1312 provides additional separation from electrode pairs 1302-1308 when in use in a container 1350.

Described thus far herein are embodiments of sensors formed from a PCB, designed to receive a fluid flow through apertures therein. However, it is expressly contemplated that sensors herein may take other shapes and configurations.

FIGS. 7A-7C illustrate a channel sensor configuration in accordance with embodiments herein. Instead of placing a sensor at an angle with respect to a flow, FIGS. 7A-7C illustrate an embodiment where a sensing area may be placed in line with fluid flow. A number of rigid or flexible sensing areas 1410 are coupled to an edge connector 1420. However, while an edge connector is shown, it is expressly contemplated that other connection mechanisms may be suitable. Sensing areas may be rigid or flexible PCBs, for example. Each sensing area 1410 is electrically isolated from adjacent sensing, as illustrated by reference numeral 1412. Sensing areas 1410 may be formed into a tube (either cylindrical, ovular or other polygonal shape) such that fluid flows past each sensing area.

Channel sensor 1400 includes an edge connector 1420 which can provide sensed electrical parameter signals to a signal reader. Edge connector 1416 may have a shielding layer 1414. Routing 1416 between edge connector 1420 and each sensing area may also be shielded.

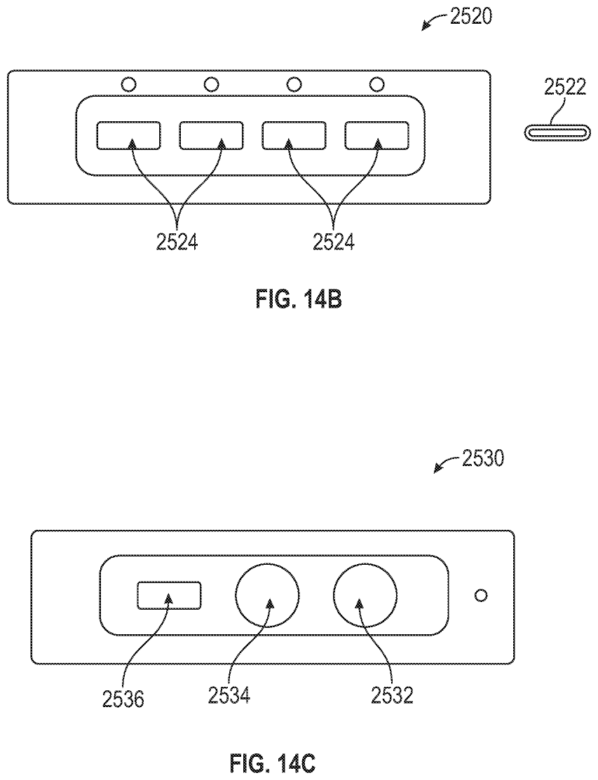

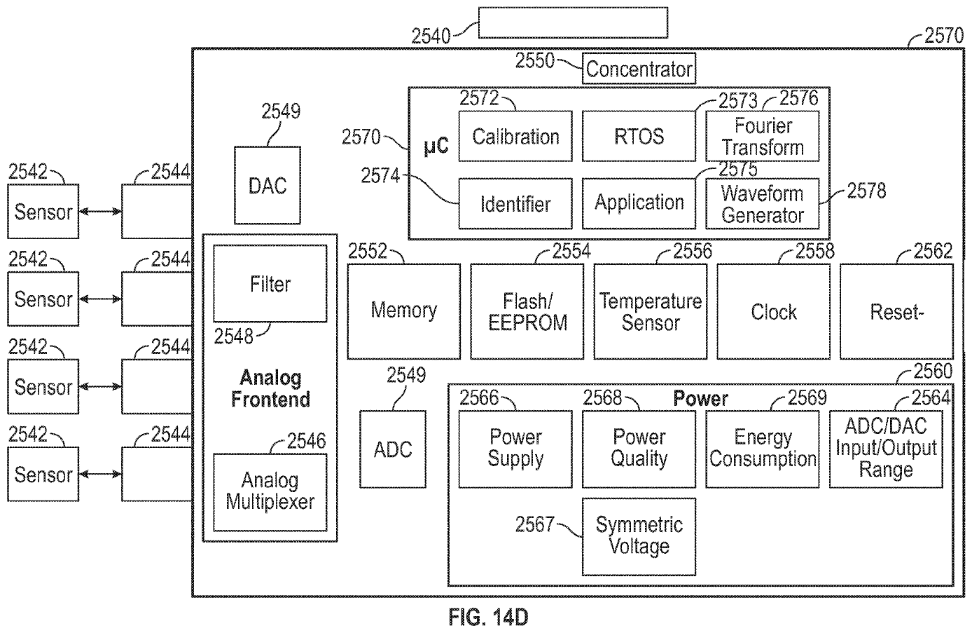

FIGS. 7B-7C illustrate cross-sectional views of channel sensor 1400. FIG. 7B illustrates a view of sensor 1400 such that material flow would be into, or out from, the page. FIG. 7C illustrates a cross sectional view along a length of sensor 1400, as indicated by line 1450. Fluid flow relative to FIG. 7C is indicated by arrow 1440. FIG. 7B can also be viewed as a cross section taken along line 1460 of FIG. 14C. As illustrated in FIG. 7B, a connection 1418 may be present between the surface and the edge connector, through the shielding layer.

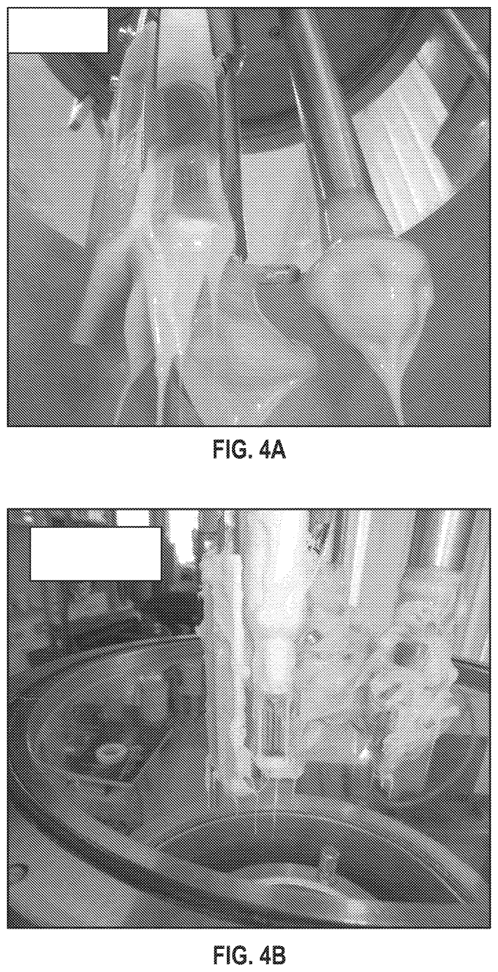

Properties of a fluid flowing through a volume formed by channel sensor 1400 can be detected using tomographic techniques. For example, any one of sensing areas 1410 may serve as a ground for a particular measurement. Sensors described in FIGS. 1-6 are limited in the number of apertures that can be machined into a PCB board. Additionally, the aperture dimensions are limited by machining abilities. The smaller the width of an aperture, the higher the signal strength. However, smaller widths make it difficult for viscous materials to flow through.

In contrast, the sensor illustrated in FIG. 7 may support a greater number of sensing areas 1410. Eight sensing areas are illustrated in FIGS. 7A-7C, however it is expressly contemplated that more sensing areas may be possible. For example, there may be as many as 10, or 12, or 16, or 20 sensing areas. Using a larger surface of a PCB, each sensing area provides a strong signal.

Like sensors described with respect to FIGS. 1-6, sensors herein allow for signal measurement, analysis and proactive or reactive changes to be made to a system in real-time or substantially real-time.

A channel sensor such as that described herein allows for direct contact between a sensing area and a material flowing past. It is possible to measure conductivity and dielectric constant directly (which may be converted, for example, to impedance).

A sensitivity for channel sensors is dependent on the diameter of the sensor, e.g. diameter 1442. In some embodiments, instead of a single channel 1400 in line with a fluid flow, there are multiple channels 1400 present. For example, multiple channels may be coplanar such that fluid flows through each simultaneously.

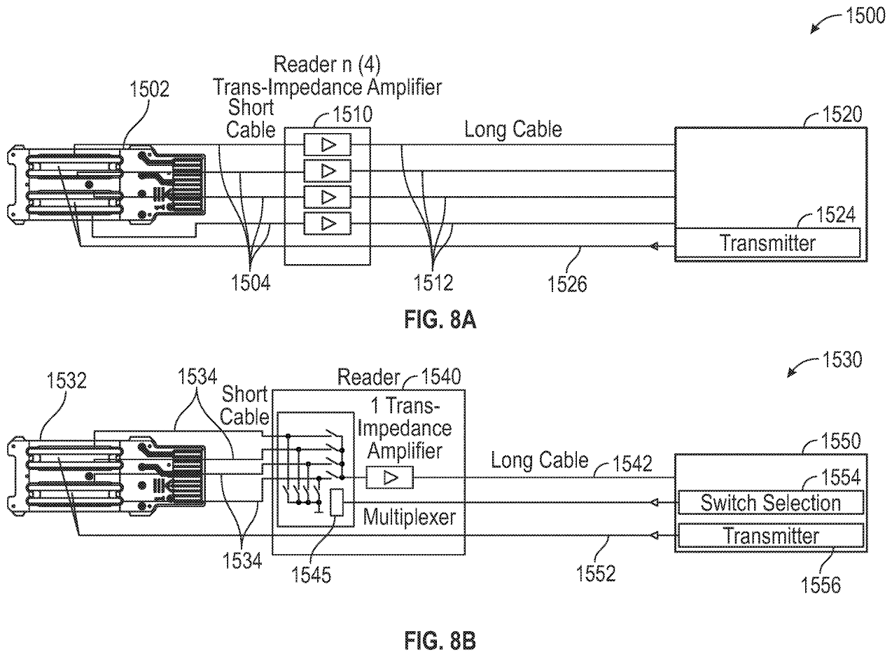

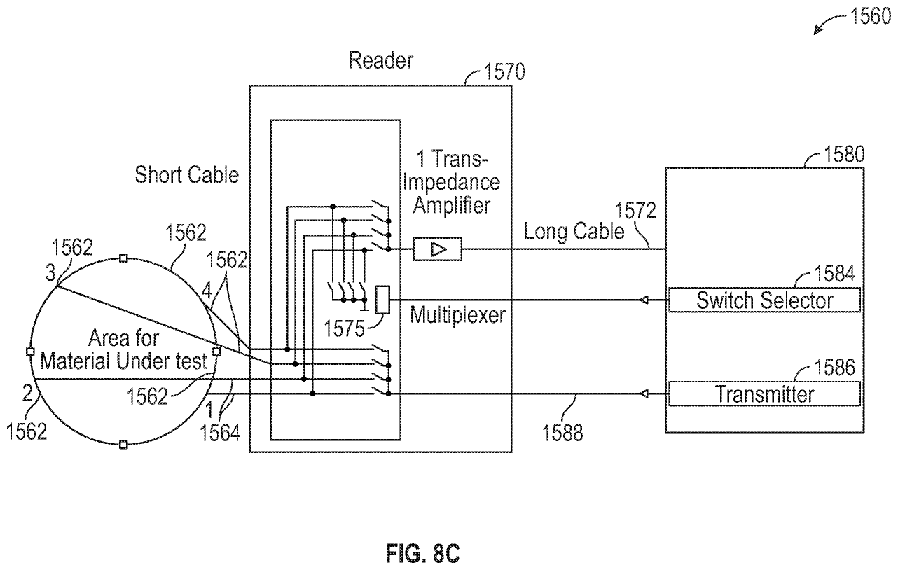

FIGS. 8A-8C illustrate electrical schematics for sensors in accordance with embodiments herein. FIG. 8A illustrates a conventional schematic, while FIGS. 8B-8C illustrates an alternative electrical schematic that may be used in embodiments herein.

Measurement system 1500 includes an example sensor 1502 that generates four sensor signals. It is expressly contemplated that systems herein may include more, or fewer, than four signal sources. Each signal is received and transferred to a signal reader 1510, using a first cable 1502. Signal reader may include a trans-impedance amplifier for each of the signal sources. The amplified signal is then communicated to a 4 channel AD converter 1520, which requires a converter for each of the four signals. A transmitter 1524 provides a voltage 1526, or voltage instruction, to be applied to transmitting electrodes on sensor 1510. Transmitter 1524 may, in some embodiments, provide a current instead of a voltage, such that the receiving electrode receives a voltage.

In FIG. 8B, signal reader 1540 includes a multiplexer 1545, which allows for selective receiving of sensor signals from sensor 1532, over cables 1534. An embodiment is illustrated where a single multiplexor 490 switches between each of the electrode pairs, such that only one trans-impedance amplifier is needed within reader 1540. However, it is expressly contemplated that a multiplexor may allow for reader 1540 to receive more than four signals and interface, for example, with multiple sensors 1532. Additionally, complexity of physical hardware is reduced as signals may be communicated to AD converter 1550, over a signal cable 1542. Only a single channel AD-converter 1550 is needed, which receives the single cable 1542, to convert a received signal. Voltage 1556 is transmitted by transmitter 1552. Multiplexor 1545 is controlled by a switch interface 1554.

FIG. 8C illustrates an electrical schematic 1560 for a channel sensor. In the embodiment illustrated in FIG. 8C, four sensing areas 1562 form a channel through which material can flow. Each allows for a signal to be received, using a cable 1564. As illustrated, signal ready 1570 has a multiplexer that can switch each of electrodes 162 from serving as a ground, transmitting or receiving electrode. In some embodiments, a multiplexer 1575 may switch electrodes 1562 such that different electrodes 1652 serve as a receiving electrode or ground during a measuring operation. Switching which electrode serves as a receiving electrode or ground may provide a more complete picture of a fluid passing through the channel sensor. Multiplexer 1575 may be controlled by a switch selector 1584.

Reader 1570 transmits signals to AD-converter 1580 using cable 1572. A voltage or current 1588 is transmitted by transmitter 1586.

FIGS. 8A-8C illustrate embodiments where four electrodes are present. However, it is expressly contemplated that more, or fewer, electrodes may be present in a given sensing system.

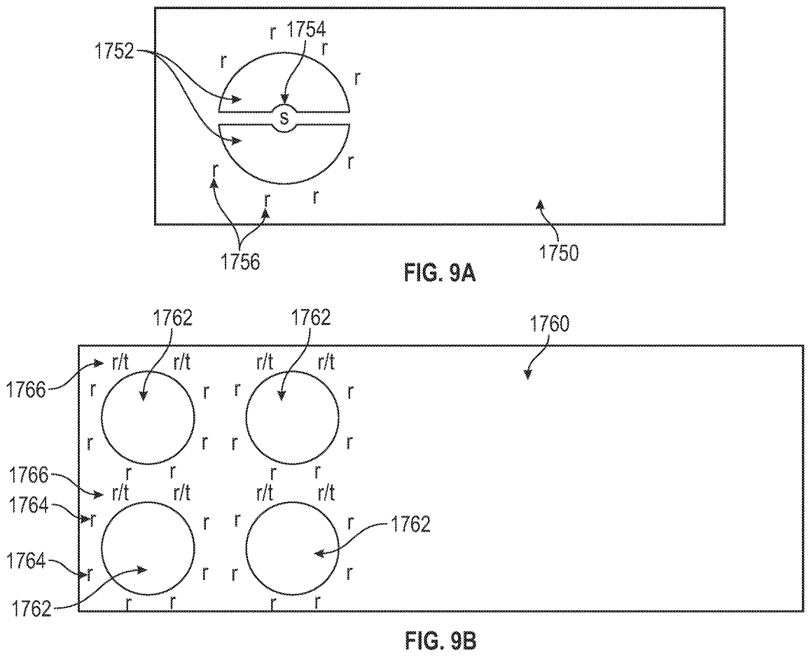

FIGS. 9A-9B illustrate embodiments of tomographic sensors. While stacking flat PCB sensors can increase sensitivity, it is noted that tomographic configurations may also have varying levels of sensitivity.

FIG. 9A illustrates an alternative channel sensor arrangement with two flow channels 1752. Sensor 1750 may include a PCB, flexible or rigid, or a different support structure. A center electrode 1754 is placed in between flow channels 1752, and a number of receiving electrodes 1756 are placed in the inner surface of sensing channels 1752. Sensing surfaces may cover some, or all, of an inner circumference of flow channels 1752. A center electrode 1752 may provide a transmitting and/or ground function, such that all of sensing areas 1756 may serve as receiving electrodes, in some embodiments. However, it is expressly contemplated that, in some embodiments, one or more of sensing areas 1756 may serve as either a transmitting electrode or a ground electrode.

FIG. 9B illustrates a channel sensor arrangement with multiple flow channels 1762. Each flow channel 1762 may be constructed similar to those illustrated in FIGS. 8A-8C, with sensing areas 1764, 1766 being angled with respect to a sensor housing 1760. Sensing areas 1764 may be in line with a fluid flow, or angled with respect to a fluid flow direction. Sensor 1760 includes a support structure may be a rigid or flexible PCB, or another suitable support structure. Flow channels 1762 each include a number of sensing areas 1764, 1766. As illustrated, many sensing areas 1765 may serve as receiving electrodes, while other sensing areas 1766 may be switched between transmitting or receiving electrodes. In some embodiments, each sensing area 1764 is capable of serving as a transmitting, receiving, or grounding electrode. In some embodiments a subset of sensing areas (e.g. only areas 1766) are configured to serve as more than a receiving electrode.

While FIGS. 9A-9B illustrate embodiments where a flow channel includes 8 sensing areas arranged in a circle, it is expressly contemplated that more sensing areas, or fewer sensing areas, may be present. For example, there may only be two sensing areas, or three sensing areas, or four sensing areas, or five sensing areas, or six sensing areas, or seven sensing areas, or there may be more than nine sensing areas, or more than ten sensing areas, or more than twelve sensing areas, or more than 16 sensing areas, or more than 20 sensing areas.

Additionally, while only four sensing channels 1762 are illustrated in FIG. 9B, it is expressly contemplated that more, or fewer, may be present. As the sensitivity of a sensing channel 1762 is inversely proportional to the diameter, it is contemplated that more sensing channels 1762, with smaller diameters, will increase sensitivity of a sensor 1760. For example, six or more channels 1762 may be present, or eight or more channels 1762, or ten or more channels 1762, or ten or more channels 1762, or more than 12 channels 1762, or more than 16 channels 1762 may be present.

Laminated structures consist of at least one non-conductive, or insulating, layer. In embodiments where the electronics are integrated onto a surface of a standard component—e.g. a molded material, a composite, etc. In such embodiments, the non-conductive layer may be formed of durolastic materials. One suitable durolastic material may include an FR-4 epoxy. However, other suitable materials may be used. In some embodiments, the non-conductive layer may be formed from a polyamide, a polycarbonate, a polypropylene, a phenolic material, ABS or another suitable material. In accordance with embodiments herein, the non-conductive layer may be modifiable to receive solder. In accordance with embodiments herein, the non-conductive layer must be modifiable to receive a conductive material—e.g. through metallization or another suitable process.

As described herein, laminated structures can be formed of a number of suitable materials. In some embodiments, laminated structures are formed using additive or subtractive manufacturing techniques. Such “printed” materials may allow for embodiments herein to be implemented in a number of additional configurations.

In some embodiments, laminate structures can be formed using classical additive manufacturing techniques—e.g. Fused filament fabrication, SLA, IJ. Non-conductive materials for such embodiments may include SLA/SLS materials, which may be UV-curable, for example. Sintermaterials, such as PA or ceramics may also be used. Fused filament fabrication materials, such as ABS or another suitable material, may also be used.

Conductive materials for laminated structures may include a base material with a surface finishing, in accordance with embodiments herein. The base material may be copper, for example. Surface finishing materials may include nickel or gold. Liquid inks may be used, and may contain silver or graphite materials. In some embodiments, nanomaterials such as graphene or carbon nanotube-based conductive inks or sprays may be used. Silver chloride may be used, for example. Carbon inks may be used, in some embodiments, either alone or as a complement to a conductive silver inks. Carbon inks may provide lubricity, protection of the silver surface and prevention of silver migration. Some conductive inks may include, for example: AgNW, AgNP, AuNP, CuNW, CuNP, PdNP, or a mixture thereof.

Dielectric inks may be used in some embodiments herein to print dielectric layers, conformal coatings and/or encapsulations. Non-conductive, dielectric inks may insulate multilayer circuitry to allow for circuitry crossover and multilayer applications. Dielectric inks offer flexibility, humidity resistance and improved strength.

Resistive inks may be used in accordance with some embodiments herein. Resistive inks may be based on blends of silver, carbon and non-conductive pigments to adjust resistance levels for printed resistors, potentiometers and heating elements.

In some embodiments herein, 3D electronic printing techniques are used, such as piezo/valve jet, aerosol based jetting, multinozzle ink jetting, 3D dispensing, printing/laser ablation, pneumatic spraying, and/or US spraying.

Using 3D printing techniques, it is possible to form sensors described in embodiments herein directly onto a surface that contacts a fluid—e.g. into a conduit, a dispenser, a mixing unit, a container, etc. A greater range of functional elements, such as flexibility, is possible. Electrically functional inks are deposited on the substrate, which results in active or passive devices.

For example, a conduit could be formed with a conductive pattern that allows for measurement of an electrical parameter. The conduct may include multiple sensing areas along its length to track electrical parameters as a fluid passes each sensing area.

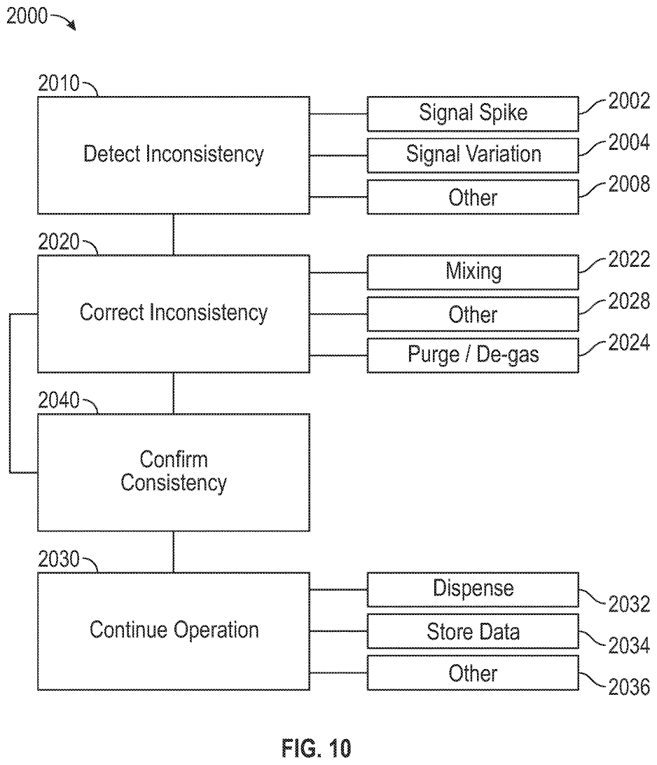

FIG. 10 illustrates a method for detecting and correcting quality concerns in a mixture in accordance with embodiments herein. Method 2000 may be practiced using sensor systems such as those described herein, combinations thereof, or other suitable sensors.

In block 2010, an inconsistency in a mixture is directed. The inconsistency may be entrained air, a mix ratio drift, inconsistent mixing-droplet formation, sedimentation, creaming- or another inconsistency from normal flow. Detection may be accomplished by detecting a spike, as illustrated in block 2002, in a sensed electrical parameter by one or more electrode pairs on a PCB sensor. Detection may also be accomplished by detecting a variation in sensed values, as illustrated in block 2004, measured between a first electrode pair and a second electrode pair of a sensor system. Other detection methods 2008 described herein may be used. Detection may occurs as a mixture flows through, or past, an electrode pair.

The electrical parameter sensor may be a disposable sensor intended to be discarded after use, in some embodiments. The sensor may include one or more pairs of electrodes in a coplanar arrangement such that the dispensed material flows through different electrode pairs. The sensor may also, or alternatively, include multiple sensing areas in an in-line arrangement such that material flow is parallel, or substantially parallel to, the sensing area. The inclusion of multiple electrode pairs of electrodes of varying sides may help to detect air bubbles or droplets of varying sizes as they flow through a sensing area.

In block 2020, the detected inconsistency is corrected. Correction may include further mixing 2022 the mixture, for example to ensure a consistent concentration, correct a detected mix ratio drift, reduce the risk of phase separation, and/or stabilize a dispersion or emulsion. Correction may also include degassing the mixture 2024, either to remove a detected air bubble or to remove entrained air introduced during a remixing step. Degassing may be accomplished using a vacuum, for example, or by purging a portion of the mixture containing the entrained air. Other suitable correction measures 2028, such as correcting a mixture composition, may also be used, such as a purge.

In some embodiments, it may be possible to mitigate a detected air bubble without purging, for example by instead sending a signal to the motor controlling fluid flow to increase speed and dispense an amount of material needed to replace the volume of air occupied by the bubble. In some embodiments, an applied pressure may increase, or a volumetric flow rate increased, in order to provide a similar volume of material if the bubble was not present.

In block 2040, consistency of the mixture may be confirmed prior to dispensing the mixture, in block 2030. For example, using sensors described herein, the consistency of the mixture may be confirmed by conductivity spikes stabilizing, e.g. reducing in severity and/or number, or by confirming that conductivity differences in electrode pairs have narrowed to an acceptable level. If consistency is not confirmed, the process may proceed back to block 2020 so that correction can be continued, or a new correction strategy may be selected.

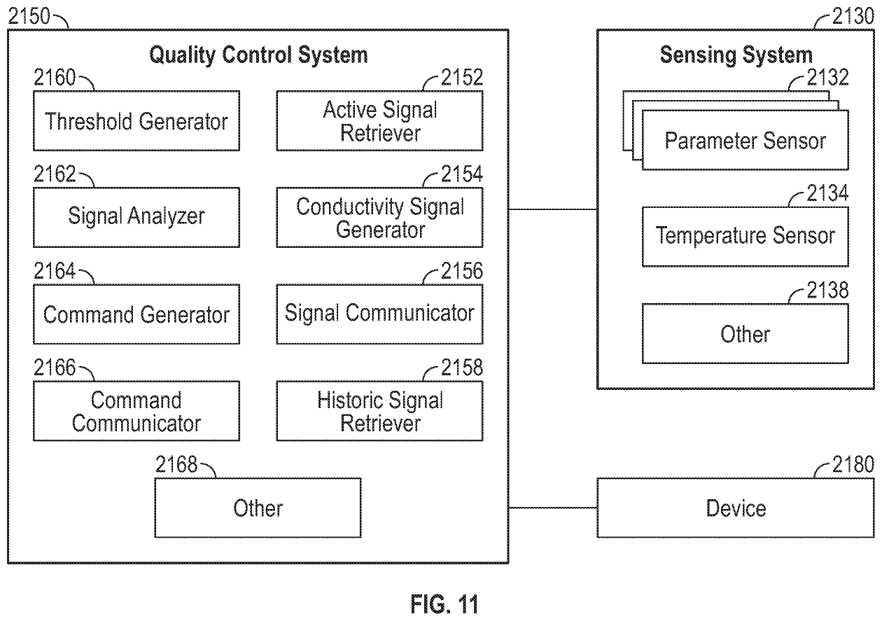

FIG. 11 illustrates a quality control system, in accordance with embodiments herein. Quality control system 2150 may be used to identify and correct a detected inconsistency in a mixture. Quality control system 2150 may be implemented in a static environment—e.g. as a dip stick or other analysis tool for a contained fluid—or a dynamic environment—e.g. in a fluid flow conduit where fluid moves through electrode pairs in a PCB board.

Some systems and methods herein may benefit from using relative thresholds instead of absolute thresholds. Base levels may be important to measure to have a more accurate relative threshold. For example, if a conductivity measurement drops below a proportionate factor to the base level (e.g. to 50% of the base level) then it can be determined that an inconsistency is present—e.g. a concentration gradient indicative of poor mixing, droplets indicative of phase separation, or entrained air. Relative thresholds may be helpful to reduce waste of material on accidental purges, or wasted time in attempting to correct an inconsistency that may not be present, or may not be at a level that requires correction.

Inconsistency detection system 2150 may be implemented by a suitable computing device in communication with a sensing system 2130. Sensing system 2130 may include one or more electrode pairs 2132 in direct contact with a material flow. Electrode pairs 2132 may be positioned such that fluid flows between them, or such that fluid contacts a surface of them. Electrode pairs 2132 may be part of a printed circuit board, for example, formed within apertures machined or built into the printed circuit board. The apertures may be closed on both ends, or open on one end, in a comb-like structure, for example. Electrode pairs 2132 may be printed onto a PCB. Printed electrode pairs 2132 may be arranged in a comb-like structure. Sensing system 2130 may also include a temperature sensor 2134. Temperature sensor 2134 may be shielded from direct contact with a material flow, in some embodiments. Sensing system 2132 may include other features 2138.

Sensor signals from sensing system 2130 are received by quality control system 2150 using an active signal retriever 2152. Active signal retriever 2152 may receive signals from sensing system 2130 periodically or continuously during an operation. Received sensor signals may be impedance signals, conductivity signals, dielectric constant signals, or a combination thereof. In embodiments where a conductivity value is used to detect an inconsistency, a conductivity signal generator 2154 may convert a received signal to a conductivity value. The signal value, and/or the conductivity value, may be provided to a data store, for example using signal communicator 2156. A similar process may be done for applications where a different electrical parameter is preferred for analysis purposes.

A historic signal retriever 2158 may communicate with a data store to retrieve previously captured signal values. Historic signal values of interest may include signal values retrieved in a recent period of time, from the same batch or mixture of materials. For example, values retrieved over a previous number of seconds or minutes may be important. In some embodiments, signal values may drift over longer periods of time due to changes in temperature, material aging, mixture ratio fluctuations, etc. But inconsistencies may be detectable as a rapid change in conductivity or a divergence of conductivity measurements in a sensing system from each other. Threshold generator 2160, in some embodiments, generates a relative threshold either periodically or continuously, based on historic signals. The relative threshold may be an absolute value, for example specifying that an increase or decrease of X % over Y time indicates an inconsistency. If conductivity values have fluctuated more significantly, the threshold change value may be larger, while if conductivity values have not fluctuated significantly, the threshold change value may be smaller.

Signal analyzer 2162 compares the received signal, or calculated conductivity, to the threshold and, if a deviation outside the allowed threshold is detected, command generator 2164 generates a command, which is communicated, using command communicator 2166, to a device 2180.

Device 2180 may, in some embodiments, include a display component, and the generated command may be an update to a graphical user interface, presented on the display component, indicating the detected inconsistency. Device 2180 may, in some embodiments, include a feedback component, such as audio, visual or haptic feedback that indicates to a controller that an air bubble is detected. Device 2180 may also be a correction mechanism, and command generator 2164 may generate a command to conduct a correction mechanism selected based on the detected inconsistency, e.g. a purge valve, a re-mixing command, a degassing command, etc.

System 2150 may include other features 2168.

In some embodiments, threshold generator includes a machine learning model to forecast the conductivity time series data into the future from historical data. This forecast may include a so-called confidence intervals. The training may be done upfront on a reference data set with no detected quality control concerns, or with quantified quality control concerns. Signal analyzer 2162 then compares a received signal to determine whether it falls within, or outside of, the confidence interval.

In some embodiments, at regular intervals (e.g., 10 ms, 100 ms, etc.), threshold generator generates a prediction for the conductivity value, with confidence bands based on the historic signals retrieved by historic signal retriever. If the actual value measured drops below a lower confidence band, or goes above a higher confidence band, signal analyzer detects an inconsistency. If the conductivity measurement is within the confidence bands, signal analyzer 2162 provides an output that no inconsistency, or no inconsistency requiring correction has been detected. Command generator 2164 may provide an indication that a GUI of device 2180 does not require updating.

A relative threshold is an important component of an air detection system because of the noise present in the data. The statistical concept of confidence bands can account for this-if data have more noise, the confidence bands are further away from the current value and the inconsistency detection algorithm will not yield wrong detections just because of noisy data, where a simple thresholding approach can suffer from this in this case.

While conductivity is discussed herein as the value of interest, it is expressly contemplated that other material parameters, such as the amount of electrical current, the relative permittivity (er) or impedance could be used instead or as well for the detections algorithm.

Measuring conductivity can provide valuable information regarding quality of a mixture. For example, as described herein, and in the Examples Section of PCT/US2022/52343, conductivity measurements may be used for determining consistency issues due to lot-to-lot variation, entrained air, droplet formation, aging, concentration gradients, dispersion separation or emulsion separation.

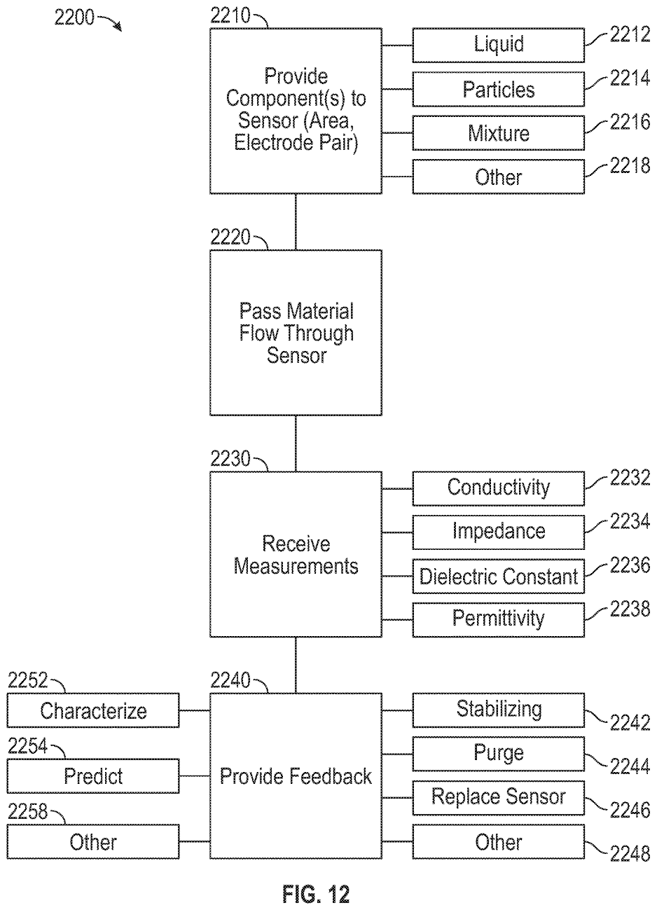

FIG. 12 illustrates a method of quality controlling a material dispensing system in accordance with embodiments herein. Method 2200 may be used with the dispensers described herein, or another suitable sensing system.

In block 2210, one or more components to be dispensed are provided to a sensing area. The sensing area may be a material dispenser, a transport line to a material dispenser, before a nozzle, atomizer, or other transportation mechanism or container within a fluid system. For example, a material dispenser may dispense a liquid 2212, particles 2214 either in suspension or otherwise. The material may also be a mixture 2216 of materials, for example an emulsion or another A and B component mixture. An emulsion must be dispensed as a stable emulsion, and reactive A:B components should be provided at a desired mix ratio. Other components 2218 may also be provided to a sensing area prior to dispensing.

In block 2220, the mixture passes through a sensing system before, for example before being dispensed, stored, removed from storage. Passing through a sensing system may entail passing through a portion of a sensing body such that the material (e.g. a mixture or a component) directly contacts a sensor. Direct contact between a material and an electrode pair ensures accurate measurements. Passing through a sensing

In block 2230, conductivity measurements are received from the sensing system. The sensing system may have multiple sensors, for example a plurality of electrode pairs that, when a sufficient voltage is passed through them, detects an electric parameter of the material. Based on the sensed parameter value, a number of things may be determined for the material. For a mixture, a mixing ratio may be determined. For a curable material, a curing progressing may be detected. Aging may also be detectable, as well as differences between batches of materials. Instability indications—such as entrained air, impending phase separation, etc. may also be detectable. Sensor measurements may be taken serially, for example one signal received every second, or more frequently. Measurements may also be taken in parallel, for example from each of a plurality of electrode pairs or sensing areas. The electrode pairs or sensing areas may be coplanar with each other, in some embodiments. Electrical parameters sensed may include conductivity 2232, impedance 2234 or dielectric constant 2236 or another suitable parameter.

In block 2240, feedback is provided based on the electrical parameter measurements. Feedback may include characterization of the material, as indicated in block 2252. For example, a mix ratio may be detected, entrained air or single component fluid pockets, an age indication or other parameter of interest may be calculated and provided. A prediction may also be provided, as indicated in block 2254. For example, based on a trend of previous conductivity sensor readings, it may be possible to predict future behavior of the material being measured. Other characterization information 2258 may also be provided. For example, a conductivity reading trending in one direction may indicate that a mix ratio is moving toward an edge of an acceptable range and, therefore, that a mix rate should be changed, or that an increase in instability is trending toward phase separation. Similarly, a conductivity reading may indicate that a curable component is curing.

Feedback may also indicate corrective action is needed. For example, an emulsion or dispersion experiencing separation may need stabilizing 2242—e.g. remixing, heating, etc. Feedback may also indicate that a purge of one component, multiple components, or a mixture, is needed, as indicated in block 2244. In embodiments where a material has corrosive effects, or cures over time, predictive feedback may provide an indication that the sensor needs to be replaced, as indicated in block 2246. Other predictive information may also be provided, as indicated in block 2238, that may trigger other actions, as indicated in block 2248.

In some embodiments, as illustrated herein, providing feedback may also include providing conductivity readings, material characterizations or predictions to a customer, controller of a dispenser, or other useful information such as material source, batch number, material name, dispensing temperature, dispensing pressure, material concentration(s), mix ratio, or any other information.

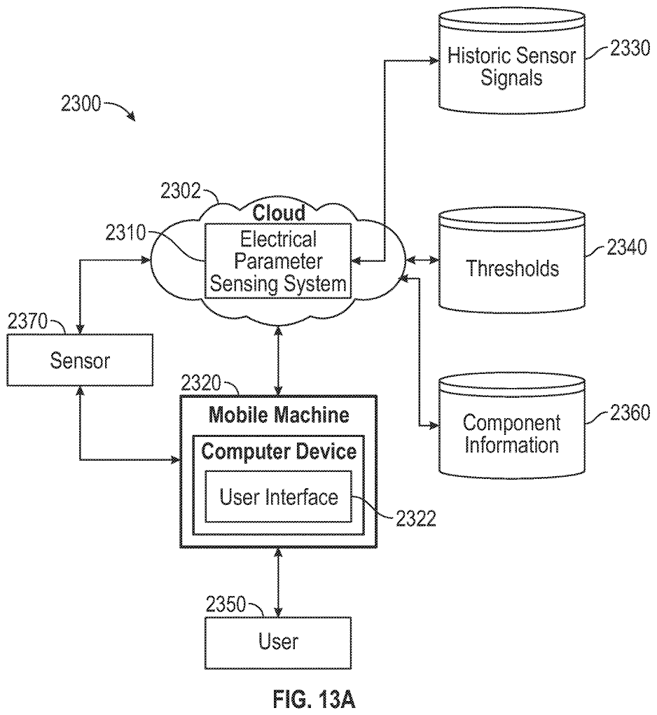

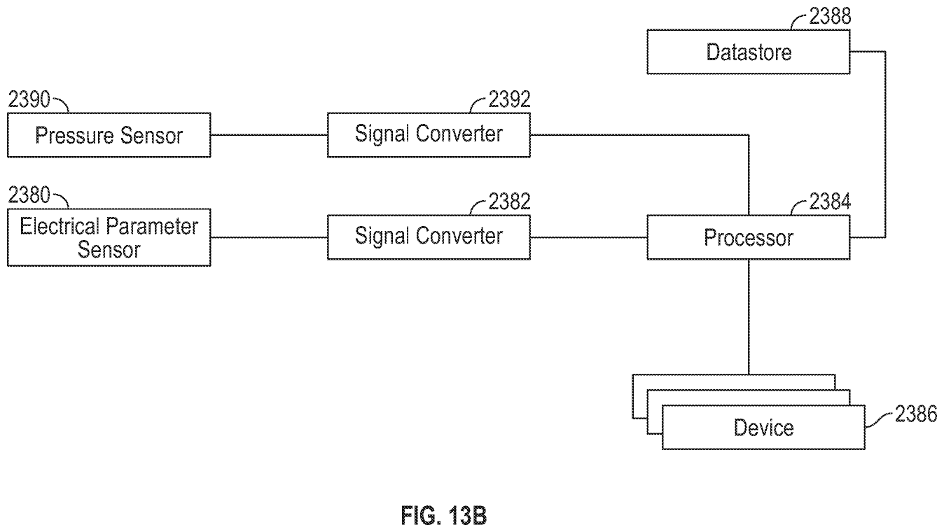

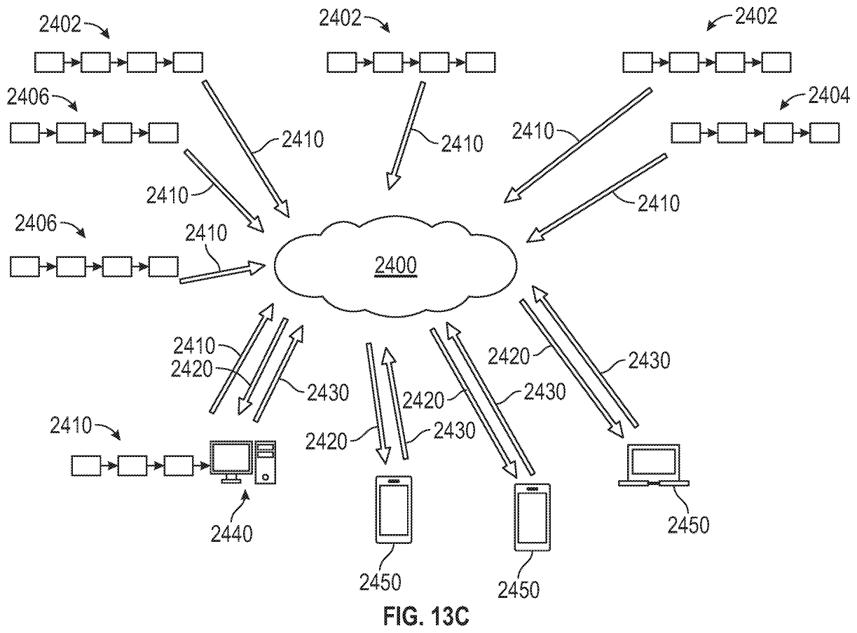

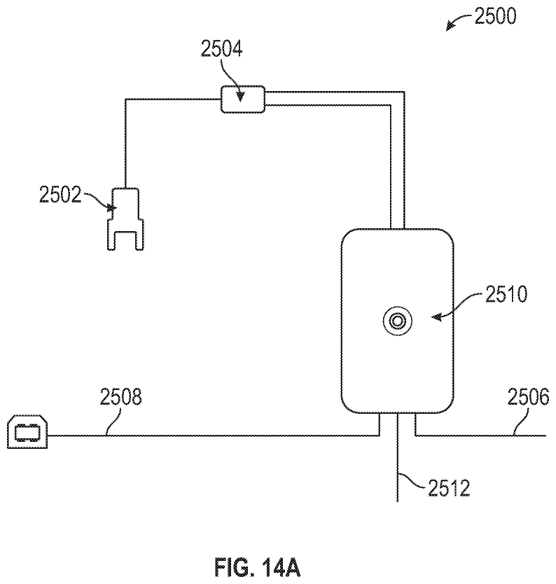

FIGS. 13A-13C illustrate a conductivity measurement system in a network of systems in accordance with embodiments herein.

In the example shown in FIG. 13A, some items are similar to those shown in earlier figures. FIG. 13A specifically shows that a conductivity sensing system 2310 can be located at a remote server location 2302. Therefore, computing device 2320 accesses those systems through remote server location 2302. User 2350 can use computing device 2320 to access user interfaces 2322 as well. For example, a user 2350 may interact with an application on the user interface 2322 of their smartphone 2320, or laptop 2320, or other computing device 2320 to receive information from a dispensing system or a quality control system.

FIG. 13A shows that it is also contemplated that some elements of systems described herein are disposed at remote server location 2302 while others are not. By way of example, data stores 2330, 2340 and/or 2360 can be disposed at a location separate from location 2302 and accessed through the remote server at location 2302. Regardless of where they are located, they can be accessed directly by computing device 2320, through a network (either a wide area network or a local area network), hosted at a remote site by a service, provided as a service, or accessed by a connection service that resides in a remote location. Also, the data can be stored in substantially any location and intermittently accessed by, or forwarded to, interested parties. For instance, physical carriers can be used instead of, or in addition to, electromagnetic wave carriers. This may allow a user 2350 to interact with system 2310 through their computing device 2360, to initiate a seal check process.

It will also be noted that the elements of systems described herein, or portions of them, can be disposed on a wide variety of different devices. Some of those devices include servers, desktop computers, laptop computers, imbedded computer, industrial controllers, tablet computers, or other mobile devices, such as palm top computers, cell phones, smart phones, multimedia players, personal digital assistants, etc.

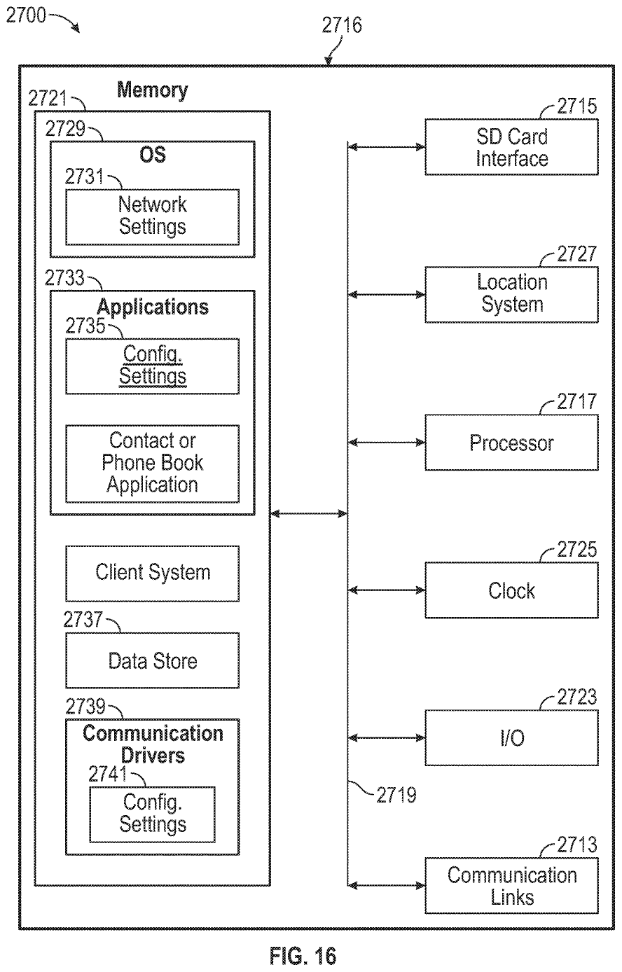

A conductivity measurement system may be any suitable system configured to, using systems and methods herein, collect conductivity measurements, conduct analysis and provide the analysis to a receiving device, storage or graphical user interface generator. FIG. 16 of PCT/US22/52343, describes operation of such a system and is hereby incorporated by reference.

System 2310 receives conductivity measurements from one or more sensors 2370. Each sensor may include one or more pairs of electrodes on a PBC. The electrodes may be coplanar and spaced similarly away from one end of the PCB, in some embodiments, or may be coplanar and in line with a length of the PCB. Sensors may be formed either by metallization or another process. Sensors 2370 are decoupled from each other such that independent conductivity signals are received from each sensor. Sensors 2370 may each include a positive and negative electrode, decoupled from one another.

Conductivity measurement systems 2310 may receive a sensor signal as a conductivity signal or a dielectric constant signal, or an impedance signal. In embodiments where a received signal is an impedance signal, a conductivity value may be calculated based on the impedance signal. similarly, a dielectric constant may be calculated based on a received impedance signal. Based on received sensor signals, calculations and/or predictions may be undertaken, as described herein. A mixing ratio may be calculated based on calibration data, stored in a datastore 2360, which may be indicative of conductivity data from pure components and/or known mixtures of components. As described above, sensors may be placed at both the inlets and outlet of a sensing zone and, therefore, system 2310 may receive sensor signals from all sensors associated with a material dispensing system. System 2310 may be configured to correct for the time delay between sensor signal capture and analysis, in some embodiments. In other embodiments, where trend information is particularly relevant, correction may not be needed.

Systems and methods are described herein that take advantage of machine learning algorithms. Machine learning models may be preferred because they can better handle noisy data, make predictions about future signal trends, and make adjustments before mix quality significantly shifts. Systems and methods described herein can calculate the mix ratio real-time. With machine learning techniques, the mix ratio could be predicted ahead of time. This allows quicker adjustments which keeps the mix ratio closer to the target value more of the time. With some current dispensers, a lot of material is entrained in the static mixer, such that, by the time a shift in mix ratio is detected, the material already in the mixer will continue to have the wrong mix ratio for at least a mixer's worth of adhesive, so identifying mix ratio issues earlier can save material and a potential purge.

Similarly, machine learning models, as described herein, may receive information from multiple systems, such as multiple sensors within a dispensing system including conductivity sensors, temperature sensors, motor speed signals, material information, etc. In some embodiment, multiple machine learning models are used simultaneously, each by an individual system such that each system's model can learn and the overall model can be improved. However, it is also expressly contemplated that non-machine learning models may also be used.

Sensing systems herein are described as having the functionality of receiving and sending communicable information to and from other devices. This may be done through an application program interface, for example, such that system 2310 can receive and communicate with pump controllers, line pressure sensors, movement controllers for portions of dispensing system, temperature sensors, heating elements, datastores having information for any of the materials being dispensed or the mixture being generated, etc.

In embodiments where machine learning models are used, datastore may also include an analyzer that learns usage behavior of a particular dispensing system in order to improve operation and predictions. Similarly, frequency and patterns of dispensing may provide information about curing and improve mixing models. For example, usage data such as frequency of dispense, purging frequency, pattern of dispense, change out of the sensor, etc., can be collected and used to train a model to more accurately predict trends and provide corrective action.

Similarly, as described herein, display 2360 may display a GUI created by generator 2320 that is updated periodically with information collected by system 2310 and/or any of datastores 2330-2360. Information may be passively updated or provided with an alert or notification as it is updated, for example current status information may be presented and an alert (visual, audio, or haptic) may be provided if the mixing ratio is drifting toward an unacceptable range. Additionally, or alternatively, notifications may be provided when a device command is generated, or when operator intervention is needed.

In some embodiments, a signal encoder and regressor may operate locally, for example using a computer processing device associated with a material dispensing system. Alternatively, either encoder or regressor, or both, may be deployed in a cloud-based storage system.

The output of encoder may be directly used to apply pressure changes on the cartridges associated with one or more material components to ensure that the mixture meets a predefined mixing ratio. E.g., if the mixed material contains too much of part A, the pressure on the cartridge containing part A is reduced and the pressure on the cartridge that contains part B increased.

A regressor may then take the encoded signals and produce a mixing ratio signal. The regressor may be a machine learning based algorithm that can be trained in any suitable way.

A first training option is a separate training option where the Encoder-Decoder model is trained on a set of signals of a variety of parts for part A, part B, and diverse mixtures. The Machine Learning Regressor is trained in a second step afterwards on the encoded signals and the corresponding mixing ratios.

A second training option is an alternating training option, where one batch of signals is used for one training step in the Encoder-Decoder and then used for one training step in the Encoder-Machine Learning Regressor part. A training step consists of a forward pass of the data in a batch, the calculation of the gradient, and an application of the gradient to optimize the weights in the model.

A third training option is a combined training option where the triplet of Encoder-Decoder pair and Machine Learning model are optimized simultaneously. This means that a batch is forward through the Encoder, and the representation obtained is forwarded through the Decoder and the Machine Learning Regressor. Then the gradients calculated with both outputs are applied in a weighted combination in the backwards pass.

Alternating or combined training may provide a benefit in that the representation of the signals is learned in a way that it has a positive effect on the performance of the Regressor which can lead to a lower error when estimating the mixing ratio. Learning a representation of signals on a variety of materials and mixing ratios also allows the models to be used on previously unseen materials of the same chemical family.

In difference to a system which only uses a single signal from the mixed material, this novel approach allows adaption for lot-to-lot variation of the raw material, where a change in one of the parts can lead to a change in the mixed signal for the same mixing ratio. It also enables tracking the mixing of the new materials of the same family be learning to fuse the signals of two parts into a mixed signal.

Data traces collected from a sensor system can be processed to provide other information as described herein. For example, sensors may provide signals that can be processed to indicate that corrective action is needed.

As described herein, in some embodiments, a sensor includes four electrode pairs. A time series of conductivity can be analyzed from the four sensor capacitors to determine when corrective action has been successful—e.g. when remixing has completed, when phase separation is reversed or a mixture has again reached stability.

For example, mixing (or remixing) may take time to reach a steady state. For example, when starting a mixing operation, backpressure and different viscosities of components can cause mixing to start off poorly and gradually stabilize. The same variance can be used to track the stabilization and indicate when the dispenser can dispense material on a workpiece or to a receiving container. The trend of the variance can be analyzed against a threshold. The threshold is specific for each material. However, instead of determining a threshold, the signal can be tested for stationarity using the Augmented Dickey-Fuller test. The advantage with this is that manual thresholds often need to be tuned for a new batch, but the ADF test is adaptable.

Inhomogeneity can also be detected using sensors described herein. The four electrode pairs should also record similar readings. Some constant offset is possible due to manufacturing tolerances, but in a stable mixing process, the variations of the four signals should be synchronous.

Once each signal has stabilized, the four sensors should have a high covariance. Negative covariance indicates a persisting anti-correlated behavior and signifies spatial inhomogeneity.

Similarly, a single component of a mixture can also be inhomogeneous, e.g., because of settling in the barrel or insufficient mixing during manufacturing. An augmented Dickey-Fuller test can again be used to confirm stationarity over a longer time. The relevant time frame would be determined by the time it takes to empty the container.

Architecture 2300 illustrates one embodiment of an implementation of a electrical parameter sensing system 2310. As an example, architecture 2300 can provide computation, software, data access, and storage services that do not require end-user knowledge of the physical location or configuration of the system that delivers the services. In various embodiments, remote servers can deliver the services over a wide area network, such as the internet, using appropriate protocols. For instance, remote servers can deliver applications over a wide area network and they can be accessed through a web browser or any other computing component. Software or components shown or described in FIGS. 1-12 as well as the corresponding data, can be stored on servers at a remote location. The computing resources in a remote server environment can be consolidated at a remote data center location or they can be dispersed. Remote server infrastructures can deliver services through shared data centers, even though they appear as a single point of access for the user. Thus, the components and functions described herein can be provided from a remote server at a remote location using a remote server architecture. Alternatively, they can be provided by a conventional server, installed on client devices directly, or in other ways.

FIG. 13B illustrates an example system architecture. In the embodiment of FIG. 13B, the system is connected through wires, such that it is not a wireless or open distributing solution. Wired communication may also be preferred in embodiments where a wireless connection would have slower transfer rates or potentially unreliability. However, as discussed with respect to FIG. 13A, it is contemplated that wireless systems may also be possible.

An electrical parameter sensor 2380 may capture an electrical parameter signal, for example from one or more PCB sensors described herein, provide that sensor signal to a signal converter 2382 where, if needed, signal conversion occurs. However, it is expressly contemplated that in some embodiments sensor 2380 may provide a sensor signal directly to processor 2384. Signal converter 2382 may convert, for example, impedance to conductivity, an analog to a digital signal, or may do another suitable conversion.

Processor 2384 receives the electrical parameter indication, and generates an electrical parameter value output, which may be provided to one or more devices 2386. Devices 2386 may include a computing device with display, a smart phone with display, a laptop with display, or to another device, for example a storage medium which stores the sensor signal for future reference. Processor 2384 may also consult one or more data stores 2388 in order to generate additional indications. For example, data store 2388 may include past conductivity sensor signals, conductivity sensor signal thresholds, commands to adjust dispensing parameters based on conductivity signal thresholds, etc. Processor 2384 may act accordingly.

In accordance with embodiments herein, system may also have a pressure sensor 2390 that generates a pressure signal, indicative of a detected pressure at a point within the dispensing system. If needed, a signal converter 2392 may convert the pressure signal from one form to another, from ampere to voltage, analog-to-digital, etc.

Processor 2384, or another suitable processor, may generate a pressure output, which may be provided to one or more devices 2386. Processor 2384 may receive signals from pressure sensor 2390 and conductivity sensor 2380 continuously throughout a process, and may be able to generate outputs continuously as well, providing substantially real-time information about a dispensing system. Processor 2384 may include one or more suitable machine learning techniques, may consult a lookup table, or perform another suitable data analysis technique on a received conductivity signal or pressure signal.

Processor 2384 may communicate with sensors 2380, 2390 wirelessly, using a wired connection, or through any other suitable network. Processor 2384 may receive signals as encrypted signals, may provide output as an encrypted output, or may operate without encryption protocols in place.

Any number of suitable communication routes are envisioned, e.g. from sensor 2390 directly to processor 2384, from sensor 2380 through signal converter 2382, and directly to datastore 2388, where it may be retrieved by processor 2384. Similarly, a request for information from devices 2386 may be sent directly to conductivity sensors 2380, 2390, to datastore 2388 or to processor 2384.

In some embodiments, an MQTT broker is used to allow, for example, devices 2386 to subscribe to a subset of data from sensor 2390 or processor 2384, for example.

In some embodiments, processor 2384 also communicates with data store 2388, such that conductivity and pressure signals are also stored for later analysis.—For example, a data set including conductivity and pressure signals over time may be used to train a machine learning algorithm, or may be used for troubleshooting purposes. For example, a machine learning algorithm may be able to detect patterns in the data set, such as an off mix ratio and need to purge, and provide indications and or thresholds about how to detect when mix ratio deviation occurs before the deviation become severe.

FIG. 13B illustrates a single processor that receives information from a single set of sensors for a dispensing operation. However, it is expressly contemplated that a production environment may have multiple dispensers running with multiple conductivity sensors and pressure sensors providing status information continuously. It is anticipated, therefore, that multiple users may want to view information about multiple production lines at the same time. FIG. 13C illustrates one configuration of a system that may be able to provide such functionality.

FIG. 13C illustrates a signal analysis system that communicates with a number of devices using a cloud-based network. As illustrated in FIG. 13C signal analysis system 2400 may communicate with a local analysis system 2440, such as that described with respect to FIG. 13B. Signal analysis system 2400 may receive a number of sensor signal data 2410 from a number of dispensing operations, such as a pilot line 2404, any of an operational line 2402, and/or a laboratory set up 2406. As described with respect to FIG. 13B, sensor signals 2400 may be digital signals, analog signals, conductivity measurement signals, pressure signals, or other signal information. For example, a low reservoir detected signal, a valve switch indication, or any other detectable indication from any of systems 2402-2406.

Signal analysis system 2400 may conduct analysis on receive sensor signal information 2400, for example using any suitable analysis tool such as lookup table, comparison thresholds, and/or machine learning algorithms to detect parameter trend information that may indicate a problem, or an action that needs to be taken, such as purging, adjusting mix ratio, etc.

Signal analysis of 2400 may provide output indicia 2420 a number of suitable devices 2450. Signal analysis system 2400 may provide output information 2120 continuously, or in response to a request 2434 information. Our request 2430 may be a one-time request for current status information, or a request to receive continuous updates going forward.

FIG. 14A-14D illustrate a sensing system in according with embodiments herein. Current sensing setups include components from different manufacturers, and data preparation and processing is done using a separate computing device. However, it is desired to have a robust and compact system that processes data quickly with limited downtime or booting time, such that quality concerns with a material are detected quickly.

In some embodiments herein a sensor contains signal preparation and processing within a single housing, e.g. a “smart” sensor. Such smart sensors contain a processing component—e.g. a microprocessor, a microcontroller, a digital signal processor or other processing circuitry. In some embodiments, a sensor also includes one or more standardized interfaces for interfacing with other systems—e.g. fieldbus systems, sensor networks, input/output links, etc. In some embodiments herein, sensor signal processing is completed without an external computer. Sensing systems herein provide decentralization, increased reliability, reduced cost, increased flexibility and simplification.

In some embodiments, a sensor system herein includes a concentrator which integrates electronic parts in a single housing. In some embodiments, all electronic components are on one PCB. In some embodiments, an analog frontend with signal conversion (e.g. AD-Converters, DA-Converters or both) are connected to a microcontroller that performs signal converting, processing and provide an output signal. Sensing systems herein may also incorporate operational circuitry, including power-supply, I/O protection circuitry, signal conditioning, reset management and/or debugging circuitry and interfaces. In some embodiments herein, the concentrator includes user-interface components such as LED signaling, UART, USB, wireless interfaces (e.g. Bluetooth®, WiFi, Zigbee®, cellular network), dot-matrix or alphanumeric display, industrial bus systems and/or tactile interface components such as push-buttons, switches, touchscreens, etc.

Systems herein may include user accessible data—e.g. a signal value, a pass/fail (e.g. “yes” or “no,” “go” or “stop,” etc.). Systems herein may provide a quality or quantity indication. Systems herein may provide a data stream with time and/or frequency-dependent data for storage and/or further processing. Systems herein may include algorithms and/or calibrations needed for data manipulation.

FIG. 14A illustrates a schematic of a sensing system in accordance with embodiments herein. Sensing system 2500 may be used with sensor described in embodiments herein, for example, or with another suitable sensor. A sensor signal reader 2502 connects to a sensor, for example an edge connector of a PCB-board that includes one or more electrode pairs. In some embodiments, a trans-impedance amplifier is present to convert current measurements to voltage. A concentrator 2510 receives sensor signals, processes said sensor signals, and provides an output. An output may be provided using an I/O device 2506 and/or another wired or wireless communication protocol 2508. A power source 2512 may provide power to concentrator 2510. While a wired power source 2512 is illustrated, it is possible that power may be provided wirelessly, or concentrator 2510 may be integrated into a material dispensing system from which it draws power.

FIG. 14B illustrates one example interface 2520 of a concentrator, that may receive sensor signals using one or more sensor signal receiving ports 2524. Other data or inputs may be received through another receiver 2522, in some embodiments.

FIG. 14C illustrates another interface 2530, which may receive a coupling to an input/output device. Power may be provided, for example using port 2434. Data may be communicated from a concentrator using a computer link 2436.

FIG. 14D illustrates a component diagram of a sensing system 2540 in accordance with embodiments herein. One or more sensors 2542 provide sensor signals, received by one or more receivers 2544 coupled to, or included within, a housing 2570. In some embodiments, system 2540 includes an analog front-end which may include a filter 2548 and/or an analog multiplexor 2546. A converter, e.g. a DA- or DC-converter 2549 may be present. Concentrator 2550 may include non-volatile memory 2552, flash memory 2554, or another suitable information storage. A temperature sensor 2556 may be incorporated into concentrator 2350, or receive a temperature signal from a temperature sensor. Concentrator 2550 may include a clock 2558. Concentrator 2562 may also include reset functionality 2562.

A sensor analyzer 2570 may include calibration data and/or functionality 2572. A real-time operating system 2573 may manage functionality. Sensor analyzer 2570 my include Fourier transformer 2576. Sensor analyzer 2570 may include a waveform generator 2576. Sensor analyzer may include other applications 2575 that provide other functionality, such as detecting of material characteristics like mix ratio, material age, curing progress, etc. Sensor analyzer 2570 may also include an identifier 2574 that identifies a type of sensor.

Concentrator 2550 may include a power management system 2560 that includes, or accesses, a power supply 2566. A power quality 2568 may be monitored. Energy consumption 2569 may be tracked. Conversion input and output ranges 2564 may be stored. A symmetric voltage 2567 may be used.

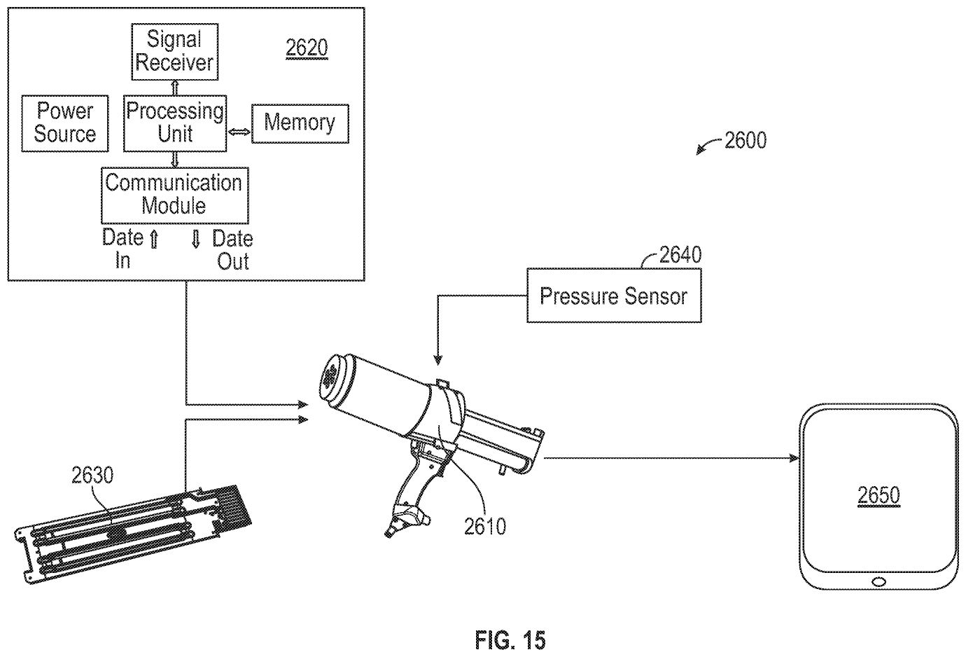

FIG. 15 illustrates a dispensing system in accordance with embodiments herein. Many dispensing operations are done with a portable, handheld system. Errors in dispensing or adhesive failure can result if material quality or machine settings are not correct. For example, an incorrect mix ratio or an incorrect pressure setting may result in an unacceptable product. It is desired to have a handheld dispensing system that can provide real-time sensing and feedback to a user. Described in FIG. 15 is one example of a system that can receive and process sensor signals without a separate computing device. Described herein are many embodiments of sensors that may be used with a dispenser. Described herein are systems for measuring pressure in a dispensing system. System 2600 includes a dispenser 2610. Dispenser 2610 is illustrated as an adhesive dispenser 2610, however other dispensers may also benefit from systems described herein. Dispenser 2610 includes an in-line sensor 2630 that senses electrical properties of a material being dispensed. A pressure sensor 2640 is incorporated into dispenser 2610 and monitors the pressure within the dispenser.

Dispenser 2610 also includes a signal processing system 2620. A signal receiver receives a sensed parameter signal from sensor 2630. A processing unit, which may include any suitable processor or processing circuitry, processes the sensed signal. A memory may store calibration data, historic signals, etc. A display 2650 may present processed information to a user, the information received from signal processing system 2620, for example using a communication module. Display 2650 may be integrated into dispenser 2610, or another display visible to a dispenser operator, such as a mobile computer, a worksite display, etc. However, while a display 2650 is illustrated as conveying processed information to an operator, it is expressly contemplated that output from signal processing system 2620 can be presented as audio or haptic feedback in some embodiments herein.

Based on sensed signals, signal processing system 2620 may also actuate a change in dispensing parameters. For example, a mix ratio may be sensed that as drifted away from a specified mix ratio. Signal processing system 2620 may, based on the sensed mix ratio drift, adjust a mix ratio by changing a pump speed for one component. Signal processing system 2620 may control pump speed directly, or indirectly, such that an instruction to change the pump speed is sent to a pump controller. Signal processing system 2620 may also communicate the mix ratio drift, e.g. through display 2650. In some embodiments, signal processing system 2620 may only communicate a detected material issue—e.g. mix ratio, aging, curing, pressure, etc.—and an operator may need to take steps to address the issue manually. However, it is expressly contemplated that, in some embodiments, dispenser parameters are adjusted automatically, in real-time, based on signals from sensors 2630, 2640.

Information about expected process parameters—e.g. mix ratio, dispensing pressure—may be detected in any suitable manner. In some embodiments, a dispenser receives expected process parameters from an NFC tag, RFID tag, or other information storage system on a material to be dispensed.

FIGS. 16-18 illustrate example devices that can be used in the embodiments shown in previous Figures. FIG. 16 illustrates an example mobile device that can be used in the embodiments shown in previous Figures. FIG. 16 is a simplified block diagram of one illustrative example of a handheld or mobile computing device that can be used as either a worker's device or a supervisor/safety officer device, for example, in which the present system (or parts of it) can be deployed. For instance, a mobile device can be deployed in the operator compartment of computing device for use in generating, processing, or displaying the data.

FIG. 16 provides a general block diagram of the components of a mobile cellular device 2716 that can run some components shown and described herein. Mobile cellular device 2716 interacts with them or runs some and interacts with some. In the device 2716, a communications link 2713 is provided that allows the handheld device to communicate with other computing devices and under some embodiments provides a channel for receiving information automatically, such as by scanning. Examples of communications link 2713 include allowing communication though one or more communication protocols, such as wireless services used to provide cellular access to a network, as well as protocols that provide local wireless connections to networks.

In other examples, applications can be received on a removable Secure Digital (SD) card that is connected to an interface 2715. Interface 2715 and communication links 2713 communicate with a processor 2717 (which can also embody a processor) along a bus 2719 that is also connected to memory 2721 and input/output (I/O) components 2723, as well as clock 2725 and location system 2727.

I/O components 2723, in one embodiment, are provided to facilitate input and output operations and the device 2716 can include input components such as buttons, touch sensors, optical sensors, microphones, touch screens, proximity sensors, accelerometers, orientation sensors and output components such as a display device, a speaker, and or a printer port. Other I/O components 2723 can be used as well.

Clock 2725 illustratively comprises a real-time clock component that outputs a time and date. It can also provide timing functions for processor 2717.

Illustratively, location system 2727 includes a component that outputs a current geographical location of device 2716. This can include, for instance, a global positioning system (GPS) receiver, a LORAN system, a dead reckoning system, a cellular triangulation system, or other positioning system. It can also include, for example, mapping software or navigation software that generates desired maps, navigation routes and other geographic functions.

Memory 2721 stores operating system 2729, network settings 2731, applications 2733, application configuration settings 2735, data store 2737, communication drivers 2739, and communication configuration settings 2741. Memory 2721 can include all types of tangible volatile and non-volatile computer-readable memory devices. It can also include computer storage media (described below). Memory 2721 stores computer readable instructions that, when executed by processor 2717, cause the processor to perform computer-implemented steps or functions according to the instructions. Processor 2717 can be activated by other components to facilitate their functionality as well. It is expressly contemplated that, while a physical memory store 2721 is illustrated as part of a device, that cloud computing options, where some data and/or processing is done using a remote service, are available.

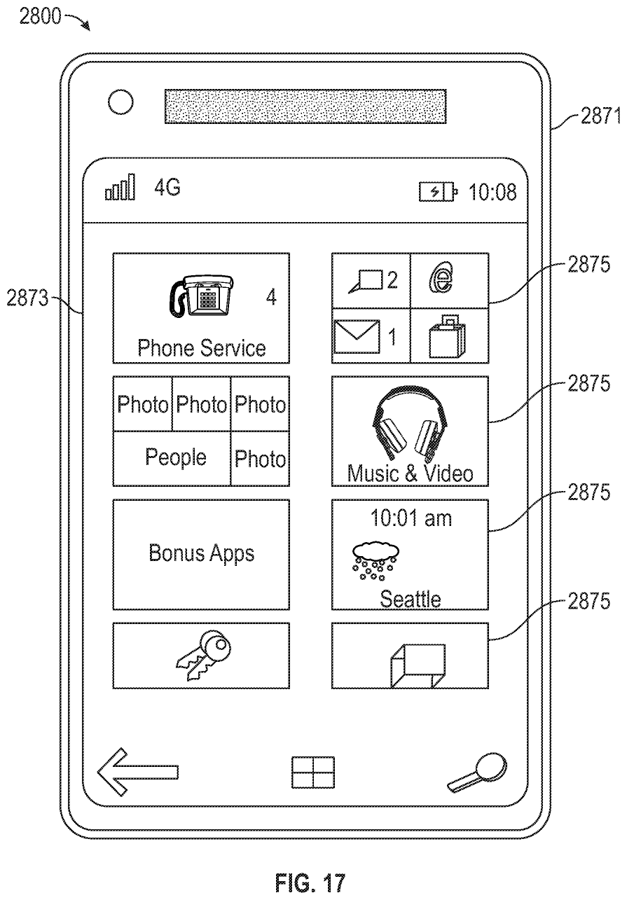

FIG. 17 shows that the device can also be a smart phone 2871. Smart phone 2871 has a touch sensitive display 2873 that displays icons or tiles or other user input mechanisms 2875. Mechanisms 2875 can be used by a user to run applications, make calls, perform data transfer operations, etc. In general, smart phone 2871 is built on a mobile operating system and offers more advanced computing capability and connectivity than a feature phone. Note that other forms of the devices are possible.

However, while FIG. 17 illustrates an embodiment where a device 2800 is a smart phone 2871, it is expressly contemplated that a display may be presented on another comping device.

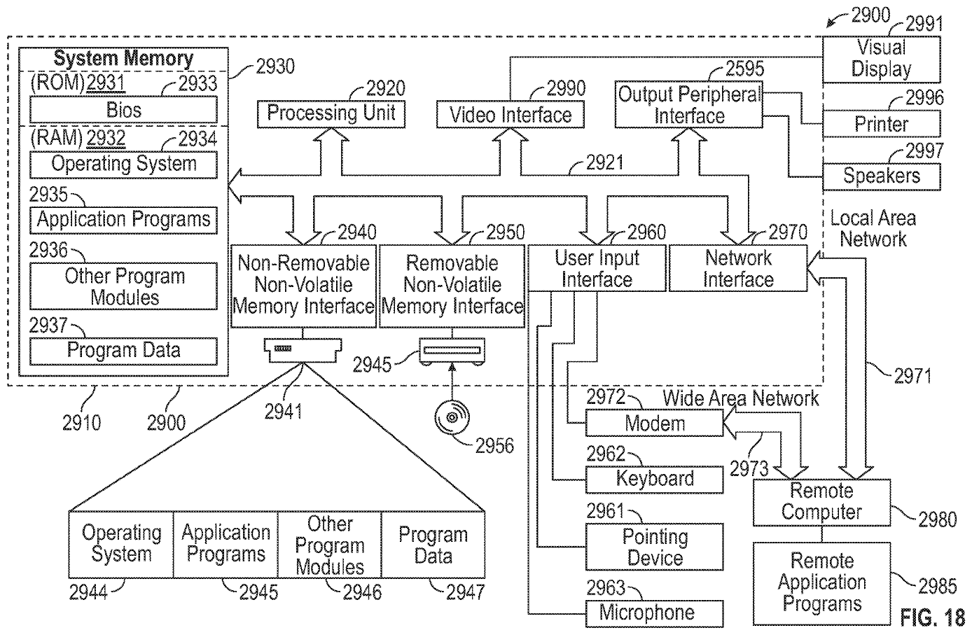

FIG. 18 is one example of a computing environment in which elements of systems and methods described herein, or parts of them (for example), can be deployed. With reference to FIG. 13A, an example system for implementing some embodiments includes a general-purpose computing device in the form of a computer 2910. Components of computer 2910 may include, but are not limited to, a processing unit 2920 (which can comprise a processor), a system memory 2930, and a system bus 2921 that couples various system components including the system memory to the processing unit 2920. The system bus 2921 may be any of several types of bus structures including a memory bus or memory controller, a peripheral bus, and a local bus using any of a variety of bus architectures. Memory and programs described with respect to systems and methods described herein can be deployed in corresponding portions of FIG. 13A.

Computer 2910 typically includes a variety of computer readable media. Computer readable media can be any available media that can be accessed by computer 2910 and includes both volatile/nonvolatile media and removable/non-removable media. By way of example, and not limitation, computer readable media may comprise computer storage media and communication media. Computer storage media is different from, and does not include, a modulated data signal or carrier wave. It includes hardware storage media including both volatile/nonvolatile and removable/non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computer 2910. Communication media may embody computer readable instructions, data structures, program modules or other data in a transport mechanism and includes any information delivery media. The term “modulated data signal” means a signal that has one or more of its characteristics set or changed in such a manner as to encode information in the signal.

The system memory 2930 includes computer storage media in the form of volatile and/or nonvolatile memory such as read only memory (ROM) 2931 and random-access memory (RAM) 2932. A basic input/output system 2933 (BIOS) containing the basic routines that help to transfer information between elements within computer 2910, such as during start-up, is typically stored in ROM 2931. RAM 2932 typically contains data and/or program modules that are immediately accessible to and/or presently being operated on by processing unit 2920. By way of example, and not limitation, FIG. 26 illustrates operating system 2934, application programs 2935, other program modules 2936, and program data 2937.

The computer 2910 may also include other removable/non-removable and volatile/nonvolatile computer storage media. By way of example only, FIG. 18 illustrates a hard disk drive 2941 that reads from or writes to non-removable, nonvolatile magnetic media, nonvolatile magnetic disk 2952, an optical disk drive 2955, and nonvolatile optical disk 2956. The hard disk drive 2941 is typically connected to the system bus 2921 through a non-removable memory interface such as interface 2940, and optical disk drive 2955 are typically connected to the system bus 2921 by a removable memory interface, such as interface 2950.

Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (e.g., ASICs), Application-specific Standard Products (e.g., ASSPs), System-on-a-chip systems (SOCs), Complex Programmable Logic Devices (CPLDs), etc.

The drives and their associated computer storage media discussed above and illustrated in FIG. 16, provide storage of computer readable instructions, data structures, program modules and other data for the computer 2910. In FIG. 18, for example, hard disk drive 2941 is illustrated as storing operating system 2944, application programs 2945, other program modules 2946, and program data 2947. Note that these components can either be the same as or different from operating system 2934, application programs 2935, other program modules 2936, and program data 2937.

A user may enter commands and information into the computer 2910 through input devices such as a keyboard 2962, a microphone 2963, and a pointing device 2961, such as a mouse, trackball or touch pad. Other input devices (not shown) may include a joystick, game pad, satellite receiver, scanner, or the like. These and other input devices are often connected to the processing unit 2920 through a user input interface 2960 that is coupled to the system bus but may be connected by other interface and bus structures. A visual display 2991 or other type of display device is also connected to the system bus 2921 via an interface, such as a video interface 2990. In addition to the monitor, computers may also include other peripheral output devices such as speakers 2997 and printer 2996, which may be connected through an output peripheral interface 2995.

The computer 2910 is operated in a networked environment using logical connections, such as a Local Area Network (LAN) or Wide Area Network (WAN) to one or more remote computers, such as a remote computer 2980.

When used in a LAN networking environment, the computer 2910 is connected to the LAN 2971 through a network interface or adapter 2970. When used in a WAN networking environment, the computer 2910 typically includes a modem 2972 or other means for establishing communications over the WAN 2973, such as the Internet. In a networked environment, program modules may be stored in a remote memory storage device. FIG. 18 illustrates, for example, that remote application programs 2985 can reside on remote computer 2980.

In the present detailed description of the preferred embodiments, reference is made to the accompanying drawings, which illustrate specific embodiments in which the invention may be practiced. The illustrated embodiments are not intended to be exhaustive of all embodiments according to the invention. It is to be understood that other embodiments may be utilized and structural or logical changes may be made without departing from the scope of the present invention. The following detailed description, therefore, is not to be taken in a limiting sense, and the scope of the present invention is defined by the appended claims.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the singular forms “a,” “an,” and “the” encompass embodiments having plural referents, unless the content clearly dictates otherwise. As used in this specification and the appended claims, the term “or” is generally employed In its sense including “and/or” unless the content clearly dictates otherwise.

Spatially related terms, including but not limited to, “proximate,” “distal,” “lower,” “upper,” “beneath,” “below,” “above,” and “on top,” if used herein, are utilized for ease of description to describe spatial relationships of an element(s) to another. Such spatially related terms encompass different orientations of the device in use or operation in addition to the particular orientations depicted in the figures and described herein. For example, if an object depicted in the figures is turned over or flipped over, portions previously described as below or beneath other elements would then be above or on top of those other elements.

As used herein, when an element, component, or layer for example is described as forming a “coincident interface” with, or being “on,” “connected to,” “coupled with,” “stacked on” or “in contact with” another element, component, or layer, it can be directly on, directly connected to, directly coupled with, directly stacked on, in direct contact with, or intervening elements, components or layers may be on, connected, coupled or in contact with the particular element, component, or layer, for example. When an element, component, or layer for example is referred to as being “directly on,” “directly connected to,” “directly coupled with,” or “directly in contact with” another element, there are no intervening elements, components or layers for example. The techniques of this disclosure may be implemented in a wide variety of computer devices, such as servers, laptop computers, desktop computers, notebook computers, tablet computers, hand-held computers, smart phones, and the like. Any components, modules or units have been described to emphasize functional aspects and do not necessarily require realization by different hardware units. The techniques described herein may also be implemented in hardware, software, firmware, or any combination thereof. Any features described as modules, units or components may be implemented together in an integrated logic device or separately as discrete but interoperable logic devices. In some cases, various features may be implemented as an integrated circuit device, such as an integrated circuit chip or chipset. Additionally, although a number of distinct modules have been described throughout this description, many of which perform unique functions, all the functions of all of the modules may be combined into a single module, or even split into further additional modules. The modules described herein are only exemplary and have been described as such for better ease of understanding.

If implemented in software, the techniques may be realized at least in part by a computer-readable medium comprising instructions that, when executed in a processor, performs one or more of the methods described above. The computer-readable medium may comprise a tangible computer-readable storage medium and may form part of a computer program product, which may include packaging materials. The computer-readable storage medium may comprise random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The computer-readable storage medium may also comprise a non-volatile storage device, such as a hard-disk, magnetic tape, a compact disk (CD), digital versatile disk (DVD), Blu-ray disk, holographic data storage media, or other non-volatile storage device.

The term “processor,” as used herein may refer to any of the foregoing structure or any other structure suitable for implementation of the techniques described herein. In addition, in some aspects, the functionality described herein may be provided within dedicated software modules or hardware modules configured for performing the techniques of this disclosure. Even if implemented in software, the techniques may use hardware such as a processor to execute the software, and a memory to store the software. In any such cases, the computers described herein may define a specific machine that is capable of executing the specific functions described herein. Also, the techniques could be fully implemented in one or more circuits or logic elements, which could also be considered a processor.

A sensing system for a mixture includes a sensing zone containing a mixture and a sensor within the sensing zone. The sensor includes a laminate structure including an insulated layer, a conductive layer and a conductive trace. The sensor includes a first sensing area and a second sensing area, the first sensing area is configured to operate as a transmitting or receiving electrode. The first and sensing areas are arranged in line with the sensing zone such that the mixture directly contacts both the first and second sensing area. A sensor signal is generated at the receiving electrode when an electric field is provided at the transmitting electrode. The system includes a communication component that communicates an electrical parameter signal based on the sensor signal.

The system may be implemented such that the sensing zone includes a conduit configured for the fluid to pass through, the conduit includes the first and second sensing areas.

The system m may be implemented such that the conduit includes a housing that receives the first and second sensing areas.

The system may be implemented such that the sensor includes a third sensing area configured to operate as a ground.

The system may be implemented such that the mixture contacts the first and second sensing areas simultaneously.

The system may be implemented such that the sensor contains a third sensing area.

The system may be implemented such that the sensor contains a fourth sensing area.

The system may be implemented such that the system includes a signal reader, and the signal reader includes a multiplexor.

The system of claim 8, the signal reader may, while the first sensing area is configured as a transmitting electrode, read a first sensor signal from the second sensing area and a second signal from the first sensing area.

The system may be implemented such that the sensing system detects a difference between the first and second sensor signals and, based on the difference, indicates an instability in the mixture.

The system may be implemented such that the instability indicates sedimentation, creaming, entrained air, droplet formation or inconsistent mixing in the mixture.

The system may be implemented such that, based on the instability, a controller generates an inconsistency correction plan.

The system may be implemented such that the inconsistency correction plan includes mixing, de-gassing, purging, or heating.

The system may be implemented such that the controller is configured to continue receiving sensor signals from the second and third sensing areas during the inconsistency correction plan.

The system may be implemented such that, to detect the instability, the controller is configured to, in situ, detect a difference between the first and second sensor signals, compare that difference to an acceptable threshold difference, and generate the instability indication if the difference exceeds the threshold difference.

The system may be implemented such that, based on a detection that the difference between the first and second sensor signals has decreased below the threshold difference, generating an indication that the instability is resolved.

The system may be implemented such that the sensing zone includes a mixing chamber that receives a first component flow and a second component flow.

The system may be implemented such that the sensing zone is within a dispenser configured to dispense the mixture.

The system may be implemented such that the electrical parameter is an impedance, a conductivity or a dielectric constant.

The system may be implemented such that the electrical parameter is indicative of a mixing ratio.

The system may be implemented such that the electrical parameter is indication of a fluid age.

The system may be implemented such that the electrical parameter is indicative of a cure progress.

The system may be implemented such that the transmitting electrode is parallel to a direction of fluid flow.

The system may be implemented such that the sensor includes a temperature sensor.

A dispensing system includes a mixing unit that receives a first fluid stream and a second fluid stream and produces a mixture and a sensor within a fluid flow stream of the dispensing system. The sensor includes a laminate structure including an insulated layer, a conductive layer and a conductive trace. The sensor also includes a first sensing area and a second sensing area, the first sensing area is configured to operate as a transmitting or receiving electrode. The first and sensing areas are arranged in line with the sensing zone such that the mixture directly contacts both the first and second sensing area. A sensor signal is generated at the receiving electrode when an electric field is provided at the transmitting electrode. The sensor generates a sensor signal indicative of the mixture. The system includes a dispenser configured to dispense the mixture and a communication component that communicates the sensor signal.

The system may be implemented such that the system includes a conduit configured for the fluid to pass through, the conduit includes the first and second sensing areas.

The system may be implemented such that the conduit includes a housing that receives the first and second sensing areas.

The system may be implemented such that the sensor includes a third sensing area configured to operate as a ground.

The system may be implemented such that the mixture contacts the first and second sensing areas simultaneously.

The system may be implemented such that the sensor contains a third sensing area.

The system may be implemented such that the sensor contains a fourth sensing area.

The system may be implemented such that the system includes a signal reader, and the signal reader includes a multiplexor.

The system may be implemented such that the signal reader may, while the first sensing area is configured as a transmitting electrode, read a first sensor signal from the second sensing area and a second signal from the first sensing area.

The system may be implemented such that the sensor is downstream of the mixing unit.

The system may be implemented such that the sensor is positioned upstream of the mixing and downstream from a first fluid source.

The system may be implemented such that the sensor is upstream of the mixing unit.

The system may be implemented such that the sensor is a first sensor, positioned downstream of the mixer, and the dispensing system includes a second sensor, positioned upstream of the mixer.

The system may include an analyzer that receives the sensor signal and provides an indication.

The system may be implemented such that the indication includes an age of the first fluid.

The system may be implemented such that the analyzer determines the indication by comparing the sensor signal to a stored sensor signal.

The system may be implemented such that the indication includes a cure progress indication of the mixture.

The system may be implemented such that the indication includes a mix ratio.

The system may include an analyzer that receives a first sensor signal from the first sensor, a second sensor signal from a second sensor, and a third sensor signal from a third sensor.

The system may be implemented such that the analyzer provides a mix ratio indication based on the received sensor signals.

The system may be implemented such that the analyzer provides a batch quality indication based on the received sensor signals.

The system may be implemented such that the analyzer provides an age indication based on the received sensor signals.

The system may be implemented such that the indication includes a mix quality across a cross section of the fluid flow.

The system may be implemented such that the analyzer determines the indication by applying a predictive model to the sensor signal.

The system may be implemented such that, based on the indication, a control signal is generated to purge the fluid flow.

The system may be implemented such that, in response to the sensor signal, a control signal is provided to a motor to adjust a motor speed of the motor.

The system may be implemented such that, in response to the sensor signal, a purge indication is communicated to a device.

The system may include a display component that receives the sensed signal and provides a visual indication of the sensed signal.

The system may be implemented such that the visual indication is a mix quality indication. The system may be implemented such that the communication component provides the sensed signal to a datastore.

The system may include a temperature sensor.

The system may be implemented such that, the temperature sensor is isolated from the fluid flow.

The system may be implemented such that the laminate structure includes four-layers.

The system may include an analyzer that receives the sensor signal and provides a mixture indication to a device.

The system may be implemented such that the analyzer, based on the sensor signal, generates the mixture indication.

The system may be implemented such that the analyzer converts the sensor signal into the mixture indication.

The system may be implemented such that the sensing zone includes a conduit and the laminate structure is printed onto an inner surface of the conduit.

A signal analysis system for a plurality of dispensing systems includes a first dispensing system with a first sensor that detects a first sensor signal from a first sensor within the first dispensing system, the first sensor includes a first sensor sensing area configured to directly contact a first fluid flowing through the first dispensing system, and the first sensor includes a first tomographic sensor and a second dispensing system with a second sensor that detects a second sensor signal from a second sensor within the second dispensing system, the second sensor includes a second sensor sensing area configured to directly contact a second fluid flowing through the second dispensing system, and the second sensor includes a second tomographic sensor. The system also includes an analyzer that receives the first and second sensor signals and generates a first sensor output and a second sensor output. The signal analysis system provides the first sensor output to a first device, and the second sensor output to a second device.

The system may be implemented such that the first sensor signal is received and first output provided in situ.

The system may be implemented such that the sensor signal is received using a wireless protocol.

The system may be implemented such that the output is communicated to the first device using a wireless protocol.

The system may be implemented such that the first sensor includes a laminate structure including an insulated later, a conductive layer and a conductive trace.

The system may be implemented such that the first sensor includes a first PCB sensor.

The system may be implemented such that the first sensor includes a first sensor second sensing area, the first sensor first area is configured to operate as a transmitting or receiving electrode.

The system may be implemented such that the first sensor includes a first sensor third sensing area, the first sensor third area is configured to operate as a receiving electrode.

The system may be implemented such that the first sensor signal includes: a first signal from the first sensor second area, generated while the first sensor first area, is configured to operate as a transmitting electrode, and a second signal from the first sensor third area, generated while the first sensor first area is configured to operate as a transmitting electrode.

The system may be implemented such that the first sensor includes a first sensor fourth sensing area.

The system may be implemented such that the analyzer includes a multiplexer.

The system may be implemented such that the first sensor output includes a subset of data obtained the first sensor signal, and the second device requested the subset of data.

The system may be implemented such that the system includes an MQTT broker that facilitates communication of the first sensor output based on the request of the second device.

An electrical property sensing system includes a sensor configured to directly contact a fluid. The sensor includes a laminate structure including an insulated layer, a conductive layer and a conductive trace. The sensor also includes a plurality of sensing areas, the plurality of sensing areas including a first sensing area configured to operate as a transmitting electrode, a second sensing area configured to operate as a first receiving electrode and a third sensing area configured to operate as a second receiving electrode. When an electric field is generated at the transmitting electrode, a first signal is generated by the first receiving electrode and a second signal is generated by the second receiving electrode. The system includes a communication component configured to: receive the first and second signals, generate a signal analysis based on the first and second signals, and generate a feedback signal based on the first and second signals, the feedback signal is generated in real-time.

The sensing system may include a housing configured to receive the sensor, the housing is configured to interact with a sensing zone.

The sensing system may be implemented such that the sensing zone includes a static fluid.

The sensing system may be implemented such that the sensing zone receives a flow of the fluid.

The sensing system may be implemented such that the first sensing area is substantially parallel to a direction of the flow.

The sensing system may be implemented such that the sensor is a first sensor, and the sensing system includes a second sensor.

The sensing system may be implemented such that the laminate structure includes a printed circuit board.

The sensing system may be implemented such that the fluid flows through a conduit, and the conduit includes the laminate structure.

The sensing system may be implemented such that the laminate structure is printed onto an inner surface of the conduit.

The sensing system may be implemented such that the laminate structure is flexible.

The sensing system may be implemented such that the feedback signal includes a mix ratio, a purge signal, a detected air bubble or an unstable mixture.

The sensing system may be implemented such that the electrical parameter is a conductivity, an impedance, or a relative permittivity.

The sensing system may be implemented such that the sensor further includes a second receiving electrode and a third receiving electrode, and the first, second and third receiving electrodes and the first transmitting electrode form a part of a conduit through with the fluid flows.

The sensing system may include a signal reader configured to receive the sensed electrical parameter.

The sensing system may be implemented such that the signal reader includes a multiplexer.

The sensing system may be implemented such that the multiplexor is configured to switch the signal reader from a first configuration to a second configuration. In the first configuration, a first set of sensor signals is received from a first plurality of receiving electrodes. In the second configuration, a second set of signals is received from a second plurality of receiving electrodes. The first set of sensing areas includes the first receiving electrode and not the second receiving electrode, and the second set of sensing areas includes the second receiving electrode and not the first receiving electrode.

The sensing system may be implemented such that the sensing system is incorporated into a dispenser, the dispenser having a pressure sensor, and the sensing system further includes processing circuitry and a memory configured to: receive a pressure signal, receive the electrical parameter value, determine, based on the pressure signal and the electrical parameter value, a setting change needed for the dispenser.

The sensing system may be implemented such that the communication component communicates the setting change to a user of the dispenser.

The sensing system may be implemented such that the communication component generates a command to change the setting.

A method of detecting an inconsistency in a fluid that includes receiving a sensed electrical parameter, using a signal reader, from a sensor, the sensor is in direct contact with the fluid. The sensor includes: a laminate structure including an insulated layer, a conductive layer and a conductive trace, a first sensing area and a second sensing area, the first sensing area is configured to operate as a transmitting or receiving electrode. The sensor includes the first and sensing areas are arranged in line with the sensing zone such that the mixture directly contacts both the first and second sensing area, and wherein a sensor signal is generated at the receiving electrode when an electric field is provided at the transmitting electrode. The method includes detecting, using a signal analyzer, based on the sensed electrical parameter, an inconsistency in the fluid, generating a correction indication for the inconsistency, and communicating the correction indication, using a communication component.

The method may be implemented such that communicating includes communicating the correction indication to a device with a display, such that the correction indication is presented on the display.

The method may be implemented such that the device includes the signal reader and the signal analyzer.

The method may be implemented such that the device includes a multiplexer.

The method may be implemented such that the sensor includes a second receiving electrode, the transmitting electrode, receiving electrode and the second receiving electrode are electrically coupled to an edge connector, and the signal reader receives the edge connector.

The method may be implemented such that the second receiving electrode, the transmitting electrode and the receiving electrode are substantially in line with a flow of fluid, such that the flow of fluid contacts the surface of the laminate structure during flow

The method may be implemented such that the steps of receiving, detecting and generating are done in real-time.

The method may be implemented such that the inconsistency includes: an amount of cure, a mix ratio, entrained air, or mix instability.

The method may be implemented such that the correction indication includes: a dispensing parameter change or a purge indication.

The method may be implemented such that the correction indication includes a command that causes a dispenser to automatically implement the correction indication.

The method may be implemented such that the sensor senses the electrical parameter value using bulk sensing techniques.

The method may be implemented such that the sensor senses the electrical parameter using surface sensing techniques.

The method may be implemented such that the sensor senses the electrical parameter using tomographic sensing techniques.

The method may be implemented such that the laminate structure is flexible.

The method may be implemented such that the laminate structure includes more receiving electrodes than transmitting electrodes.

The method may be implemented such that the laminate structure includes an aperture and the aperture includes the transmitting and receiving electrodes.

The method may be implemented such that the sensed electrical parameter includes an impedance, a conductivity, or a dielectric constant.

The method may be implemented such that the fluid is an adhesive.

1. A sensing system for a mixture comprising:
a sensing zone containing a mixture;
a sensor within the sensing zone, the sensor comprising:
a laminate structure comprising an insulated layer, a conductive layer and a conductive trace;
a first sensing area and a second sensing area, wherein the first sensing area is configured to operate as a transmitting or receiving electrode;
wherein the first and sensing areas are arranged in line with the sensing zone such that the mixture directly contacts both the first and second sensing area; and wherein a sensor signal is generated at the receiving electrode when an electric field is provided at the transmitting electrode; and
a communication component that communicates an electrical parameter signal based on the sensor signal.

2. The system of claim 1, and wherein the sensing zone comprises a conduit configured for the fluid to pass through, wherein the conduit comprises the first and second sensing areas.

3. The system of claim 2, wherein the conduit comprises a housing that receives the first and second sensing areas.

4. The system of claim 1, wherein the sensor comprises a third sensing area configured to operate as a ground.

5. The system of claim 1, wherein the mixture contacts the first and second sensing areas simultaneously.

6. The system of claim 1, wherein the sensor contains a third sensing area.

7. The system of claim 6, wherein the sensor contains a fourth sensing area.

8. The system of claim 6, wherein the system comprises a signal reader, and wherein the signal reader comprises a multiplexor.

9. The system of claim 8, wherein the signal reader may, while the first sensing area is configured as a transmitting electrode, read a first sensor signal from the second sensing area and a second signal from the first sensing area.

10. The system of claim 9, wherein the sensing system detects a difference between the first and second sensor signals and, based on the difference, indicates an instability in the mixture.

11. The system of claim 10, wherein the instability indicates sedimentation, creaming, entrained air, droplet formation or inconsistent mixing in the mixture.

12. The system of claim 10, wherein, based on the instability, a controller generates an inconsistency correction plan.

13. The system of claim 9, to detect the instability, the controller is configured to, in situ, detect a difference between the first and second sensor signals, compare that difference to an acceptable threshold difference, and generate the instability indication if the difference exceeds the threshold difference.

14. The system of claim 13, and wherein, based on a detection that the difference between the first and second sensor signals has decreased below the threshold difference, generating an indication that the instability is resolved.

15. The system of any of claim 1, wherein the electrical parameter is an impedance, a conductivity or a dielectric constant.

16. The system of any of claim 1, wherein the electrical parameter is indicative of a mixing ratio, a fluid age, or a cure progress.

17. The system of any of claim 1, wherein the transmitting electrode is parallel to a direction of fluid flow.

18. A signal analysis system for a plurality of dispensing systems, the system comprising:
a first dispensing system with a first sensor that detects a first sensor signal from a first sensor within the first dispensing system, wherein the first sensor comprises a first sensor sensing area configured to directly contact a first fluid flowing through the first dispensing system, and wherein the first sensor comprises a first tomographic sensor;
a second dispensing system with a second sensor that detects a second sensor signal from a second sensor within the second dispensing system, wherein the second sensor comprises a second sensor sensing area configured to directly contact a second fluid flowing through the second dispensing system, and wherein the second sensor comprises a second tomographic sensor;
an analyzer that receives the first and second sensor signals and generates a first sensor output and a second sensor output; and
wherein the signal analysis system provides the first sensor output to a first device, and the second sensor output to a second device.

19. The system of claim 18, wherein the first sensor signal is received and the first output provided in situ.

20-27. (canceled)

28. A method of detecting an inconsistency in a fluid, the method comprising:
receiving a sensed electrical parameter, using a signal reader, from a sensor, wherein the sensor is in direct contact with the fluid, and wherein the sensor comprises:
a laminate structure comprising an insulated layer, a conductive layer and a conductive trace;
a first sensing area and a second sensing area, wherein the first sensing area is configured to operate as a transmitting or receiving electrode;
wherein the first and sensing areas are arranged in line with the sensing zone such that the mixture directly contacts both the first and second sensing area; and wherein a sensor signal is generated at the receiving electrode when an electric field is provided at the transmitting electrode; and
detecting, using a signal analyzer, based on the sensed electrical parameter, an inconsistency in the fluid;
generating a correction indication for the inconsistency; and
communicating the correction indication, using a communication component.

29-40. (canceled)
